SEQUENCE LISTING (7 sequences)

SEQ ID NO: 1 - RNA, 2372 nt, synthetic construct
  source 1..2372: mol_type = other RNA
aggaataaactagtattcttctggtccccacagactcagagagaacccgccacccggggtcgccgtcctacacattgttgtgacgtgcggcccagattcgaatctgtaataaaagttttcttttcttctatatcctcagattggcagtgagaggagattttgttcgtggtgtaggctggcctactgggtggggttgggatccggactgaatccgtagaatttctgtacaacataccatggcacaccaccaccaccaccaccacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgataatagaccggtatcgtagtcgcacgacctggacacgccgaacttccgcgggtcggctgacgtcgatggggtgcgtgcaaaaaagacctacgaagccagagttcgttccagtgtgaaagtgcacatcacgagttgtgccaatgcacgttgcatcgagggctgaagccgtcttaatatagacggcacctgaagagtgattgattcgtctagaaatagacgaatcatgctgatctcaggtgctcacttgattaagacggctgtttatctcgatgctcgccctcttggcacaatcgaacttgtgcacttcagcacgggaacgaactctggcttcgtaggtcaaaaaagcacgcaagcatgtaacgtcagcctaacgcttgaagttcgcaggtgtgaggtcgtgcatgtggtctaccggttctgcttgtctacttgtacagctcgtccatgccagatcagcacccggcgaaaaaaacgaaccgtgaggtagccatgtagatactctgctcgttgccttgccgtatcagggctcaccaggagctcaggtagtggttgtcgggcagtcacactgagccgtcgatacatgctctgttctaaaaaaagtggtcgcactatagcacgctgtctcgtacatggttgtcagaagtcgcgaagttcaccttgatgccgttctctattatcagggccatgatcaagtgactgtggcttacctcacagtactcaaaaaagtgccccaagatttgaccgtccgatgaacttacgatgcccttcagctcgatgcggtggactggggtgtcgccactcgtgatgacctcgtaagcgttactgtagttttatagtgccttgaagaaaaaagtgcgctcttgatcagagccttcgggcatggcggacttgggcggatctggctgcttggactacgacgggtagcataaacaggactgcacgagagtatcgagggtggcaaatcttgtgggccaaaaaacgggcagcttgccggtggtgcaaccttgaagaagggtcagggtctttggggtggcgaacgtgcatccctcgcacacacctgtgaacttttgtacgagcacgtcgcgtgatcaagtgaccaggatgggcccgggtaatagaaaaaaaacccgggtgagctggagcctcggtggcctagcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 2330 nt, synthetic construct
  source 1..2330: mol_type = other RNA
aggaataaactagtattcttctggtccccacagactcagagagaacccgccaccaggagccgccgccagtggagggccgggcgctgcggccgcggccggggcgggcgcagggccgagcggacgggggggcgcgggccccccgggaggccgcggccactcccccccgggccggcgcggcgggggaggcggaggaccatggcacaccaccaccaccaccaccacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgataatagaccggtatcgtagtcgcacgacctggacacgccgaacttccgcgggtcggctgacgtcgatggggtgcgtgcaaaaaagacctacgaagccagagttcgttccagtgtgaaagtgcacatcacgagttgtgccaatgcacgttgcatcgagggctgaagccgtcttaatatagacggcacctgaagagtgattgattcgtctagaaatagacgaatcatgctgatctcaggtgctcacttgattaagacggctgtttatctcgatgctcgccctcttggcacaatcgaacttgtgcacttcagcacgggaacgaactctggcttcgtaggtcaaaaaagcacgcaagcatgtaacgtcagcctaacgcttgaagttcgcaggtgtgaggtcgtgcatgtggtctaccggttctgcttgtctacttgtacagctcgtccatgccagatcagcacccggcgaaaaaaacgaaccgtgaggtagccatgtagatactctgctcgttgccttgccgtatcagggctcaccaggagctcaggtagtggttgtcgggcagtcacactgagccgtcgatacatgctctgttctaaaaaaagtggtcgcactatagcacgctgtctcgtacatggttgtcagaagtcgcgaagttcaccttgatgccgttctctattatcagggccatgatcaagtgactgtggcttacctcacagtactcaaaaaagtgccccaagatttgaccgtccgatgaacttacgatgcccttcagctcgatgcggtggactggggtgtcgccactcgtgatgacctcgtaagcgttactgtagttttatagtgccttgaagaaaaaagtgcgctcttgatcagagccttcgggcatggcggacttgggcggatctggctgcttggactacgacgggtagcataaacaggactgcacgagagtatcgagggtggcaaatcttgtgggccaaaaaacgggcagcttgccggtggtgcaaccttgaagaagggtcagggtctttggggtggcgaacgtgcatccctcgcacacacctgtgaacttttgtacgagcacgtcgcgtgatcaagtgaccaggatgggcccgggtaatagaaaaaaaacccgggtgagctggagcctcggtggcctagcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - RNA, 2163 nt, synthetic construct
  source 1..2163: mol_type = other RNA
aggaataaactagtattcttctggtccccacagactcagagagaacccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgataatagaccggtatcgtagtcgcacgacctggacacgccgaacttccgcgggtcggctgacgtcgatggggtgcgtgcaaaaaagacctacgaagccagagttcgttccagtgtgaaagtgcacatcacgagttgtgccaatgcacgttgcatcgagggctgaagccgtcttaatatagacggcacctgaagagtgattgattcgtctagaaatagacgaatcatgctgatctcaggtgctcacttgattaagacggctgtttatctcgatgctcgccctcttggcacaatcgaacttgtgcacttcagcacgggaacgaactctggcttcgtaggtcaaaaaagcacgcaagcatgtaacgtcagcctaacgcttgaagttcgcaggtgtgaggtcgtgcatgtggtctaccggttctgcttgtctacttgtacagctcgtccatgccagatcagcacccggcgaaaaaaacgaaccgtgaggtagccatgtagatactctgctcgttgccttgccgtatcagggctcaccaggagctcaggtagtggttgtcgggcagtcacactgagccgtcgatacatgctctgttctaaaaaaagtggtcgcactatagcacgctgtctcgtacatggttgtcagaagtcgcgaagttcaccttgatgccgttctctattatcagggccatgatcaagtgactgtggcttacctcacagtactcaaaaaagtgccccaagatttgaccgtccgatgaacttacgatgcccttcagctcgatgcggtggactggggtgtcgccactcgtgatgacctcgtaagcgttactgtagttttatagtgccttgaagaaaaaagtgcgctcttgatcagagccttcgggcatggcggacttgggcggatctggctgcttggactacgacgggtagcataaacaggactgcacgagagtatcgagggtggcaaatcttgtgggccaaaaaacgggcagcttgccggtggtgcaaccttgaagaagggtcagggtctttggggtggcgaacgtgcatccctcgcacacacctgtgaacttttgtacgagcacgtcgcgtgatcaagtgaccaggatgggcccgggtaatagaaaaaaaacccgggtgagctggagcctcggtggcctagcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 2111 nt, synthetic construct
  source 1..2111: mol_type = other RNA
aggaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagtgagaagttgccatcgtagtcgcacgacctggacacgccgaacttccgcgggtcggctgacgtcgatggggtgcgtgcaaaaaagacctacgaagccagagttcgttccagtgtgaaagtgcacatcacgagttgtgccaatgcacgttgcatcgagggctgaagccgtcttaatatagacggcacctgaagagtgattgattcgtctagaaatagacgaatcatgctgatctcaggtgctcacttgattaagacggctgtttatctcgatgctcgccctcttggcacaatcgaacttgtgcacttcagcacgggaacgaactctggcttcgtaggtcaaaaaagcacgcaagcatgtaacgtcagcctaacgcttgaagttcgcaggtgtgaggtcgtgcatgtggtctggcaactctgcttgtttacttgtacagctcgtccatgccggatcagcacccggcgaaaaaaacgaactgtgaggtaaccatgtggatactcttctcgttggcttgccgtctcagggctcaccaggtgctcaggtagtggttgtcgggcagtcacactgggccgtcgctacatgctgtgttctaaaaaaagtggtcgcactatagcacgctgcctcgtacaatgttgtgagaagtcgtgaagttcaccttgatgccgttctttctcactacggccatgatcaagtgattgtggcttacctcactgtactcaaaaaagtgccccaagatttggccgtcctatgaactttcgatgcccttcagctcgatgcggtggactggggtgtcgcccctcgtgatcacctcggaagcgttattgtagttgtatagtgccttgaagaaaaaagtgcgctcttgatcatagccttcgggcatggcggacttgagcggatcttgctgcttcgactacgaggggtagcataaacagcactgcacgagagtatccagggtggcaaatcttgtgggccaaaaaacgggcagcttgccggtggtgcagccttgaagcagggtcagggtctttgaggtggcaaacgtgcagccctcgcccacacctgtgaacttgtgtacgagtacgtcgcctgatcaagcgaccaggatgggcccggggcggccgcttaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - RNA, 2114 nt, synthetic construct
  source 1..2114: mol_type = other RNA
aggaggaaataagagagaaaagaagagtaagaagaaatataagagccaccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaatagtgattatatcgggccccgtcgaggacacgaaaaaagaaggtgctgttagttggacaggtactatcatctcaagtcgatagtccaagtaggtttgaaccatgcatagcttgtatcaggtcatcgccaccttcattaggtgtctgatcagggaatcgcaaaaaacataccgacttccattatgggacacgtcgcttattcttggtaagtagaagttgccatcgtagtcgcacgacctacacacctcgaacttcatcatgttggctgacgtactaacagtgcgtgcaaaaaagacctacgaagccagagttcgttccagtgtgaaagtgcacatcacgagttgtgccaatgcacgttgcatcgagatttggtcccgtcttaaagggagggccacctgaatggaagttgattcgtctagaaatagacgaatcatgctgatctcaggtgctcacttgattaagacggctgtttatctcgatgcattctgaattggcacaaccgtccgagtgcacttatgatagtgaacgaactctggcttcgtaggtcaaaaaagcacgcaagcatgtaacgtcagcctaacgcttgaagttcgcaggtgtgaggtcgtgctttttcgatggcaacttggttactccaagaataagcgacgtgtcccatagatcagcacggtatgaaaaaagcgattcgtgaggtagacacctagatactctggcgatgacttacggacagctatgcgcgatgccaacctacttggactatcgacttgagtcacactgacctgtccatacatgctcaccttcaaaaaacgtgtccgcactatagcccgtcatctcgtacaaggaatgctacaccaccgccgccagccggtcactccgttgctctacttagggaacagatcaagtgactcggggttacctcacagctcgtaaaaaattttgtcaagatttgacagggtgaatcctttagtgatctttttgccgtcgaacactatggttcagtcatacgactcgtgatgatagtctaagcgttacggcgtaattatagtgcctaccagaaaaaaatagtgtcttgatcagttaaagtgatgatcatccacaggggttcgcacgttaaaaaggactacgacctggcccataaacaggccgctcagagagtatcgcgggatgcaaatcttgatgtcgaaaaaaagcccattttcaccgctcaggaacctgttacagatcggagctgatacagccccgagaacgtgcatcaaactgacacacctgtcctgttttgtacgagcgtccagggtgatcaagtctgtctgtcgccgcggccgcttaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 2111 nt, synthetic construct
  source 1..2111: mol_type = other RNA
aggaaataagagagaaaagaagagtaagaagaaatataagagccaccatggcaggaaaacctattcctaatccattattaggtttagattctacagaggccgcagcagctaaggggatcctgggcttcgtgtttaccctcgctgcctacaaatctgctattgtgacattaacctatgctgcctacaacacaactcctattgttcatttaaaggctgcctacacacttcaagatgtttctttagaagtagctgcctactatgtgcatgaaggaattagaacatatgctgcctaccaagttgattattacggcctttattatgctgcctacaatttagacactgcttctacaacattagctgcctacgttcttttatgtgtatgtctgttgattgctgcctacaaattgcctcaactttgtacagaattagctgcctacaccatacatgatattatccttgaatgtgttgctgcctacattatactggaatgcgtttactgtaaagctgcctactttgcctttagagatctttgtattgttgctgcctacaccacacttgagcaacaatataataaagctgcctactatatgttagatttgcaacctgaaacaactgctgcctacagagcccattataatattgtgacatttgctgcctaccttttaatgggaacattaggtattgtttgtcccatatgttctcaaaaatgataaggaatcgcaaaaaacataccgacttccattatgggacacgtcgcttattcttggtaagtagaagttgccatcgtagtcgcacgacctgagggtaacgaacttccacataaaggctgacgtcagttgtttgcgtgcaaaaaagacctacgaagccagagttcgttccagtgtgaaagtgcacatcacgagttgtgccaatgcacgttgcatcgagcatgcacaccgtcttaactttacaggcacctgaatggaagttgattcgtctagaaatagacgaatcatgctgatctcaggtgctcacttgattaagacggctgtttatctcgatgcgcagatttttggcacaagcaatcaagtgcacttatgggctcgaacgaactctggcttcgtaggtcaaaaaagcacgcaagcatgtaacgtcagcctaacgcttgaagttcgcaggtgtgaggtcgtgctatgttcttggcaactaaaggcaaccaagaataagcgacgtgtcccatagatcagcacggtatgaaaaaagcgattcgtgaggtattttgagagatactctgacaaacaaggttaatggttcccatgaggcaataggcagcaaatgtcacaatattatatcacactgtgtaggcagtacatgcttcaggttaaaaaactaacatgcactatacagctttatctcgtacatgctcaagcaacttggaggcagcaacaatacaaagatctcttctacttaagtaggcagtcaagtgataaacgcatacctcactaatgtaaaaaaaaacacataagatttgaatatcatgtgttgtataggcagctaattctgtacaaagtttaaaaggtttgtaggcactcgtgatcagacataaagcgttaagaacgtagtatagtgctgttgtaaaaaaagtgtctacttgatcagcagcataataaaggccgtaataattgtggtgttaggcagcagactacgaaattccttataaacagtagtaggcaagagtatctaaagaaacaaatcttagtgtgtaaaaaacctttaaatgaacaataggagttgtatggtgggcagcatatacctaattcacaataaacgtgcagtaggcagccacacctgacacgaagtgtacgagccccttagctgatcaagcctctgtagaatccccggggcggccgcttaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 2974 bp, synthetic construct
  source 1..2974: mol_type = other DNA
atggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataaggaggaaataagagagaaaagaagagtaagaagaaatataagagccaccatggaaaaaaaaaacccggggcggccgcttaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctcgagtcgacctaggatcccttctactgggcggttttatggacagcaagcgaaccggaattgccagctggggcgccctctggtaaggttgggaagccctgcaaagtaaactggatggctttcttgccgccaaggatctg

(19) United States
(12) Patent Application Publication
(10) Pub. No.: US 2026/0224679 A1
(43) Pub. Date: Aug. 6, 2026

(54) SWITCHABLE mRNA EXPRESSION PLATFORM

(71) Applicants: Petr SULC, Scottsdale, AZ (US); Mark KNAPPENBERGER, Mesa, AZ (US); Archana KIKLA, Tempe, AZ (US); Matthen SAMPLE, Tempe, AZ (US); Karen ANDERSON, Scottsdale, AZ (US)

(72) Inventors: Petr SULC, Scottsdale, AZ (US); Mark KNAPPENBERGER, Mesa, AZ (US); Archana KIKLA, Tempe, AZ (US); Matthen SAMPLE, Tempe, AZ (US); Karen ANDERSON, Scottsdale, AZ (US)

(21) Appl. No.: 19/423,282

(22) Filed: Dec. 17, 2025

Related U.S. Application Data
(60) Provisional application No. 63/735,654, filed on Dec. 18, 2024.

(51) Int. Cl. A61K 39/00; C07K 14/005; C07K 14/705; C12N 7/00; C12N 15/85

(52) U.S. Cl. CPC A61K 39/001129; C07K 14/005; C07K 14/70575; C07K 14/70596; C12N 7/00; C12N 15/85; A61K 2039/5258; A61K 2039/53; A61K 2039/55555; C12N 2710/20022; C12N 2710/20023; C12N 2710/20034; C12N 2760/16022; C12N 2760/16023; C12N 2760/16034; C12N 2770/20022; C12N 2770/20023; C12N 2770/20034; C12N 2830/50

(57) ABSTRACT

Described herein is a gene delivery system exhibiting high stability, compactness, and structural utility. The delivery system has a built-in ON switch, which only expresses the gene only in cells that contain a specified “trigger” RNA molecule, which then enables the translation of the delivered construct.

### CROSS-REFERENCE TO RELATED APPLICATIONS

This application claims priority to U.S. Provisional Patent Application No. 63/735,654 filed on Dec. 18, 2024, which is incorporated by reference herein in its entirety.

### FEDERALLY SPONSORED RESEARCH

This invention was made with government support under R01 GM145916 awarded by the National Institutes of Health. The government has certain rights in the invention.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “208192-0029-US02_sequence_listing_xml_10 Dec. 2025.xml,” was created on Dec. 10, 2025, contains 7 sequences, has a file size of 22.8 kilobytes (23,362 bytes), and is incorporated by reference in its entirety into the specification.

### BACKGROUND

RNA vaccines have recently become mainstream but have been in development for over 20 years. They still suffer from several drawbacks, such as high-dosing requirements (approx. 100 ug), and the inability to conditionally express them only the cell which they enter meets certain conditions, such as presence of disease specific RNA.

What is needed are compositions and method for delivering mRNA vaccines with the ability to activate expression in vivo.

### SUMMARY

One embodiment described herein is a nanoparticle composition comprising a folded single-stranded nucleic acid nanostructure comprising: a 5′-untranslated region (5′-UTR); a coding sequence; a structuring sequence that is complementary to the coding sequence and the 5′-UTR or a region of the coding sequence and the 5′-UTR; a 3′-untranslated region (3′-UTR); and a polyadenosine (poly-A) tail; wherein the structuring sequence is bound to the 5′-UTR and encapsulated in the nanostructure. In one aspect, the coding sequence comprises a viral nucleic acid sequence, a bacterial nucleic acid sequence, or a parasitic nucleic acid sequence. In another aspect, the viral nucleic acid sequence is derived from SARS CoV2, influenza virus, or HPV. In another aspect, the coding sequence comprises a nucleic acid encoding one or more of CD40L, CD70, TLR4, and a neoantigen. In another aspect, the coding sequence comprises a reporter gene selected from the group consisting of NanoLuc luciferase (Nluc), mCitrine, mCherry, mStrawberry, green fluorescent protein (GFP), enhanced green fluorescent protein (EGFP), yellow fluorescent protein (YFP), and red fluorescent protein (RFP). In another aspect, the nucleic acid nanostructure comprises messenger RNA (mRNA), single-stranded RNA (ssRNA), single-stranded DNA (ssDNA), or a combination thereof. In another aspect, the structuring sequence comprises one or more double crossover motifs, paranemic crossover motifs, kissing loops, or combinations thereof. In another aspect, the composition comprises a plurality of folded nucleic acid nanostructures stacked in a multimer via one or more kissing loops. In another aspect, the nucleic acid nanostructure further comprises one or more DNA or RNA staple strands. In another aspect, the nucleic acid nanostructure has a substantially cylindrical tube shape. In another aspect, the structuring sequence comprises a long hairpin along the inside edge of the cylindrical tube. In another aspect, the nucleic acid nanostructure has intramolecular folding capabilities without the need for helper nucleic acid strands. In another aspect, the structuring sequence is bound to the 5′-UTR or region of the 5′-UTR, wherein the 5′-UTR cannot bind to a ribosome or be degraded by a nuclease. In another aspect, the poly-A tail is located before the structuring sequence, after the structuring sequence, or at the 3′-end. In another aspect, one or more of the 5′-UTR and the 3′-UTR is derived from murine beta-globin. In another aspect, the 5′-UTR has a sequence that recruits RNA Helicase A (RHA). In another aspect, the sequence that recruits RNA Helicase A is from HIV 5′ leader sequence or human junD.

Another embodiment described herein is a method for selectively expressing a coding sequence in a target cell, the method comprising: delivering to a target cell a nanoparticle composition comprising a folded single-stranded nucleic acid nanostructure comprising: a 5′-untranslated region (UTR); a coding sequence; a structuring sequence that is complementary to the coding sequence and 5′-UTR or a region of the coding sequence and the 5′-UTR; a 3′-UTR; and a polyadenosine (poly-A) tail; wherein the structuring sequence is bound to the 5′-UTR and encapsulated in the nanostructure; wherein upon delivering the nanoparticle composition to the target cell, a target cell nucleic acid molecule binds the structuring sequence and displaces the 5′-UTR, thereby releasing the 5′-UTR for translation and expression of the coding sequence.

In another aspect, the method may include one or more of the following features. The target cell nucleic acid molecule may be associated with a virus or a disease. The target cell nucleic acid molecule may be associated with a cancer, a viral infection, a bacterial infection, or a parasitic infection. The nanoparticle composition may comprise a plurality of therapeutic mRNAs. The plurality of therapeutic mRNAs may be stacked in a multimer via one or more kissing loops for stoichiometric co-delivery. The target cell may be a tumor cell, a virus-infected cell, a microorganism-infected cell, a parasite-infected cell, an autoimmune cell, or a cell expressing a specific biomarker. The delivering step may comprise administering the nanoparticle composition via injection, inhalation, oral administration, or topical application.

Another embodiment described herein is a kit. In one aspect, the kit comprises the nanoparticle composition described above, a device for administering the composition, a label or instructions for use, and packaging. In another aspect, the device for administering may comprise a syringe, nebulizer, or lipid nanoparticle delivery system. In another aspect, the instructions for use may specify conditions for thermal annealing of the nanoparticle composition at specific temperatures or temperature ranges.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an overview of the design pipepline: computational design is used for the sequence structuring strand that folds the mRNA into a compact nanoparticle, which is validated with a coarse-grained model called oxRNA.

FIG. 2 shows kissing loop interactions that are used to stack different mRNAs into a multimer for stochiometric codelivery. The stacked mRNAs can also be used for conditional activation (using the same strand displacement mechanism as in FIG. 5A-B), where the 5′-UTR is placed inside the two sheets and is only accessible after release by a trigger strand.

FIG. 3A-C show designed kissing loops to fold mRNA into a tube. FIG. 3A shows an unfolded structure. FIG. 3B shows a folded structure. FIG. 3C shows a view of FIG. 3B from the front.

FIG. 4 shows a schematic of using strand displacement for conditional release of 5′-UTR in folded mRNA particle.

FIG. 5A-B show the strand displacement mechanism used to release 5′-UTR. FIG. 5A shows the Structure of OFF state of the mRNA nanoparticle, where the 5′-UTR is not accessible, the gene cannot express. FIG. 5B shows an encounter with a trigger strand (a specific RNA sequence present in the targeted cell type) releases the 5′-UTR so that the gene can be expressed.

FIG. 6 shows an agarose gel Electrophoretic Mobility Shift Assay (EMSA) of eGFP-OG nanostructures with (lanes 2-4) and without (lane 1) thermally annealing in several different buffer conditions.

FIG. 7 shows representative gel EMSA shifts of 2 eGFP-OG templates (same sequence derived from different colonies) in native and folded states. Additionally, this gel shows that other synthetic cap analogs (CleanCap® AG-3MeO, Trilink Biotechnologies) can also be utilized without any difference in the IVT or folding results.

FIG. 8 shows representative atomic force micrograph depicting folded mRNA-OG particles (rectangles) under optimized buffer and annealing conditions. The Figure is representative of folded particles encoding eGFP, nanoluciferase, and HPV epitopes.

FIG. 9 shows brightfield and GFP microscopy images of lipofectamine only (−ve control), 1 μg unfolded eGFP-OG, 1 μg thermally annealed eGFP-OG, and 1 μg Trilink eGFP mRNA (+ve control). Cells were transfected using MessengerMax™ Lipofectamine™ transfection reagent (Thermo Fisher) and imaged 48 hours after transfection.

FIG. 10 shows flow cytometry was used to measure transfection efficiency for HEK293T cells lipofected with 1 μg natively structured or thermally annealed eGFP-OG with or without the presence of RNAse Inhibitor (SUPERase OUT, ThermoFisher). Transfection efficiency is similar between the folded and unfolded groups, indicating eGFP expression from folded nanostructures.

FIG. 11A-B shows flow cytometry results for HEK293T, A549 (RNase H K/O), and A549 (Protein Kinase R K/D) transfected with folded eGFP-OG with one of two structuring strands (SSv6, same structure as all other data in this disclosure, and ACS Nano, alternative structuring strand only shown in this figure). Transfection efficiency is shown in FIG. 11A and mean brightness is shown in FIG. 11B. −ve controls indicated lipofection with no RNA present.

FIG. 12 shows the encapsulation efficiency of various eGFP coding mRNAs into MessengerMax™ Lipofectamine™ transfection reagent (Thermo Fisher). eGFP-OG Unfolded to Folded structural transition demonstrates significant increase in encapsulation, outperforming even commercially available eGFP mRNA. EGFP-OG folded corresponds to design shown in FIG. 1, and the results show its higher loading capacity for lipid nanoparticle encapsulation

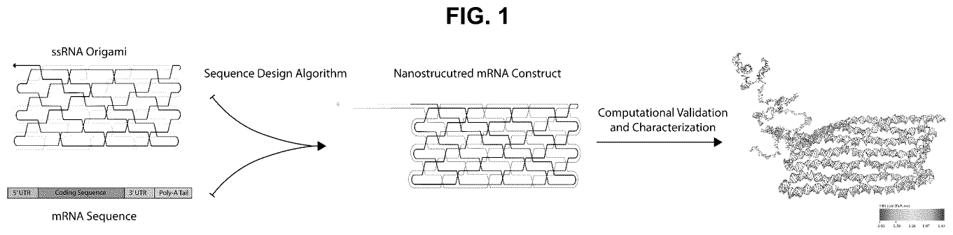

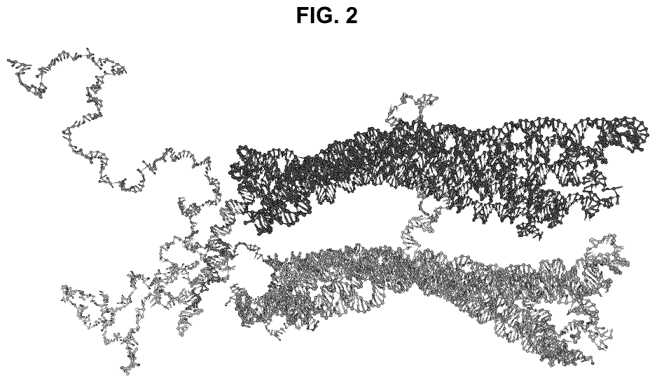

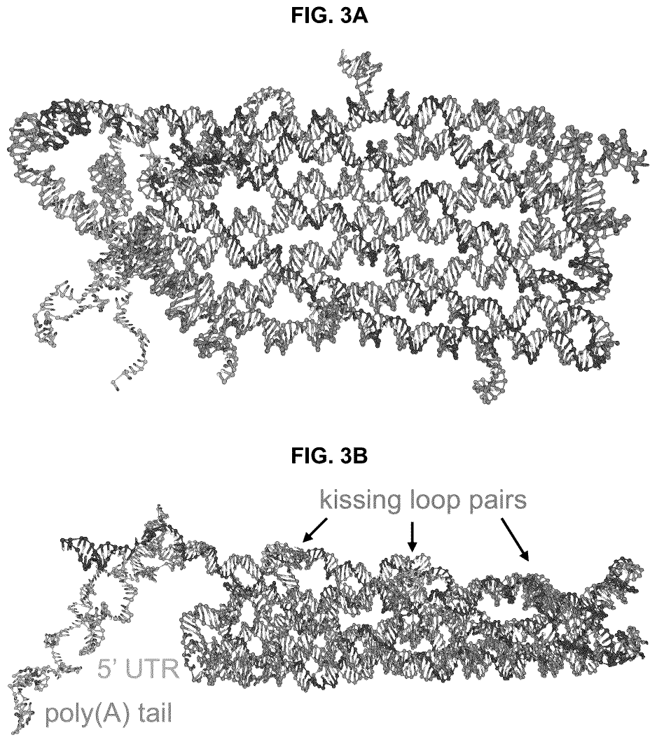

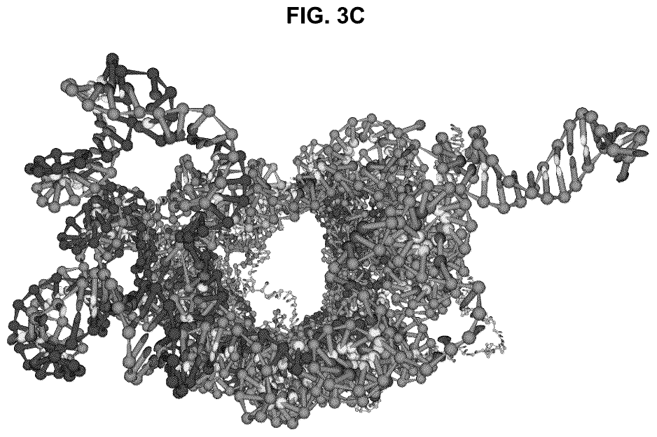

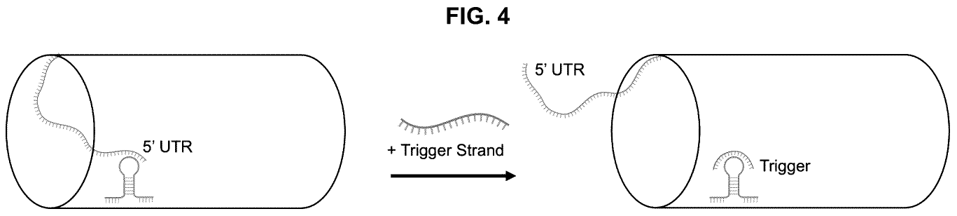

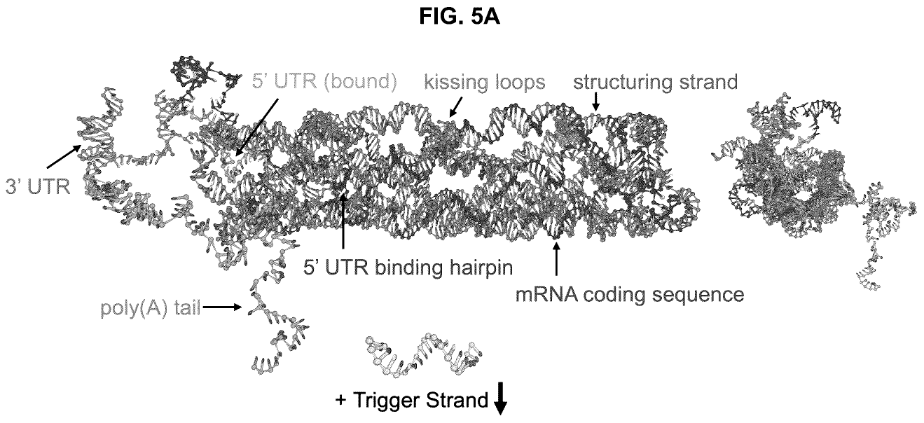

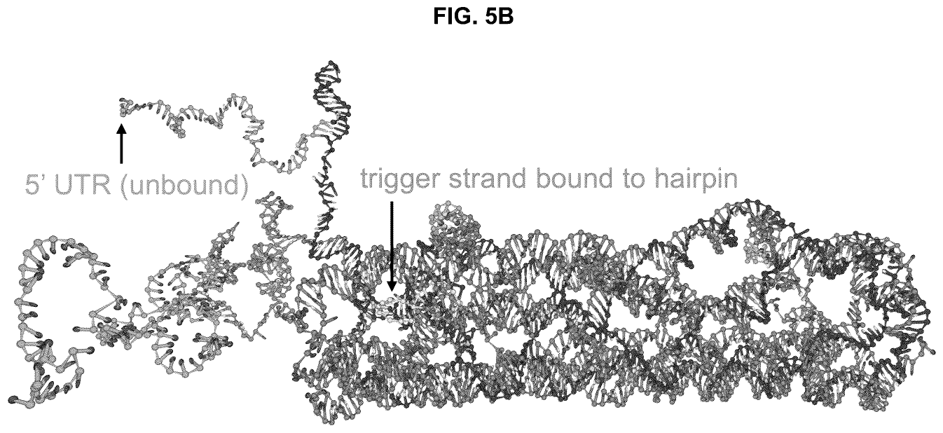

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ~20° C., ~22° C., ~25° C., or ~27° C., at standard atmospheric pressure.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof; (b) nucleotide sequences, or degenerate, homologous, or codon-optimized variants thereof, encoding polypeptides having the amino acid sequences in SEQ ID NO: 1-7; and (c) nucleotide sequences capable of hybridizing to the complement of any of the nucleotide sequences in (a) or (b).

By a polynucleotide having a nucleotide sequence at least, for example, 90-99% “identical” to a reference nucleotide sequence encoding a polypeptide is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding the polypeptide.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:4 82-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986).

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequence shown in SEQ ID NO: 1-7, or degenerate, homologous, or codon-optimized variants thereof.

The polynucleotides described herein include those encoding mutations, variations, substitutions, additions, deletions, and particular examples of the polypeptides described herein. For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that proteins are surprisingly tolerant of amino acid substitutions.

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by the method or the means described herein

Another embodiment described herein is the use of an effective amount of a nucleotide having at least 90-99% identity to SEQ ID NO: 1-7 or having the sequence of SEQ ID NO: 1-7.

Another embodiment described herein is a research tool comprising nucleotide having at least 90-99% identity to SEQ ID NO: 1-7 or having the sequence of SEQ ID NO: 1-7 described herein.

Another embodiment described herein is a reagent comprising a nucleotide having at least 90-99% identity to SEQ ID NO: 1-7 or having the sequence of SEQ ID NO: 1-7 described herein.

### Nucleic Acid Nanostructure Compositions

Disclosed herein are compositions comprising one or more nucleic acid nanostructures. A nucleic acid nanostructure may be a folded single-stranded nucleic acid nanostructure. The nucleic acid nanostructures disclosed herein comprise messenger RNA (mRNA), single-stranded RNA (ssRNA), single-stranded DNA (ssDNA), or a combination thereof.

A nucleic acid nanostructure comprises a coding sequence. The coding sequence may comprise a viral nucleic acid sequence. The coding sequence may comprise a whole gene. The coding sequence may comprise a whole gene encoding the SARS CoV2 spike protein. The coding sequence may comprise viral oncogenes. The coding sequence may comprise HPV16 E6 mRNA. The coding sequence may comprise HPV16 E7 mRNA. The coding sequence may comprise serial minigenes. The serial minigenes may encode T cell epitopes. The serial minigenes may encode the Flu M1 protein. The coding sequence may comprise a custom Influenza M1-EGFP construct. The custom Influenza M1-EGFP construct may stimulate CD8+ T cells. The coding sequence may comprise sequences encoding CD40L, CD70, and TLR4. The coding sequence may further comprise a neoantigen mRNA.

The coding sequence may comprise a bacterial nucleic acid sequence. The coding sequence may comprise a nucleic acid sequence associated with a parasite. The coding sequence may comprise a nucleic acid sequence associated with a cancer. The coding sequence may comprise a non-coding RNA sequence.

The coding sequence may comprise a reporter gene. The reporter gene may be a gene selected from the group consisting of NanoLuc luciferase (Nluc), mCitrine, mCherry, mStrawberry, green fluorescent protein (GFP), enhanced green fluorescent protein (EGFP), yellow fluorescent protein (YFP), and red fluorescent protein (RFP). The coding sequence may comprise a Kozak consensus sequence. The coding sequence may comprise a start codon. The coding sequence may comprise a V5-tag.

The nucleic acid nanostructures disclosed herein comprise a structuring sequence. The structuring sequence may be complementary to the coding sequence and the 5′-UTR. The structuring sequence may be complementary to a region of the coding sequence and the 5′-UTR. The structuring sequence may be bound to the 5′-UTR. The structuring sequence may be encapsulated in the nanostructure.

The structuring sequence may comprise one or more double (DX) crossover motifs. The structuring sequence may comprise paranemic (PX) crossover motifs. The structuring sequence may comprise a combination of double crossover motifs and paranemic crossover motifs. The structuring sequence may comprise one to nine DX or PX motifs. Different embodiments of the structuring sequence may differ in the number of crossover motifs, the relative spacing of crossover motifs to each other, and the location of crossover motifs relative to the ribosome-binding sequence (RBS) and start codon (RBS-proximal, mid-CDS, and 3′-proximal).

The structuring sequence may comprise kissing loops. The structuring sequence may comprise a combination of double crossover motifs, paranemic crossover motifs, and kissing loops. Compositions comprising multiple nucleic acid nanostructures comprising kissing loop hairpins may form complexes comprising protected areas between the nucleic acid nanostructures. The protected areas in the complexes may be used to conceal one or more UTRs and protect the UTRs from degradation prior to transfection. A kissing loop may comprise a first loop sequence which is complementary to a second loop sequence of another nucleic acid nanostructure. Compositions disclosed herein may comprise a plurality of folded nucleic acid nanostructures. The plurality of folded nucleic acid nanostructures may be stacked in a multimer via one or more kissing loops.

A nucleic acid nanostructure may comprise a 5′-untranslated region (UTR). A nucleic acid nanostructure may comprise a 3′-UTR. One or more UTRs may be short synthetic UTRs derived from murine beta-globin. The 5′-UTR may have a sequence that recruits RNA Helicase A (RHA). The 5′-UTR may be a 5′-UTR isolated from the HIV 5′ leader sequence which specifically recruits RHA. The 5′-UTR may be a 5′-UTR from human junD that recruits RHA. The 5′-UTR may be a 5′-synthetic murine beta-globin UTR. The 3′-UTR may be a 3′-synthetic murine globin UTR.

A nucleic acid nanostructure may comprise a polyadenosine (poly-A) tail. The location of the poly-A tail may affect the translation initiation. The placement of the poly-A tail before or after the structuring sequence may affect the eIF4G-mediated scaffolding of poly-A-binding protein (PABP) and eIF4A, and thereby influence mRNA circularization and translation initiation. The poly-A tail may be a defined 142-nt poly(A) tail positioned at the mRNAs 3′-end. Some embodiments described herein may be modified such that both the 3′-UTR and poly-A tail are repositioned to an unstructured loop upstream of the structuring sequence.

A nucleic acid nanostructure may comprise one or more DNA or RNA staple strands.

A nucleic acid nanostructure may have a substantially cylindrical tube shape. Nucleic acid nanostructures with a substantially cylindrical tube shape may have a long hairpin embedded in the structuring sequence. The long hairpin may be located on an inside edge of the cylinder. The long hairpin may contain a loop sequence complementary to the first 16 nucleotides of the 5′-UTR.

A nucleic acid nanostructure may have intramolecular folding capabilities without the need for helper nucleic acid strands. A pair of complementary sequences within the structuring sequence and the 5′-UTR may bind during the folding of the nucleic acid nanostructure. Binding of the 5′-UTR to the structuring sequence may prevent the binding of a ribosome to the 5′ UTR. Binding of the 5′-UTR to the structuring sequence may protect the 5′-UTR from nuclease degradation. Binding and release of the 5′-UTR may depend on the length of the long hairpin.

The switchable mRNA expression platform described herein provides an approach to nucleic acid therapeutics through the use of folded single-stranded nucleic acid nanostructures that enable conditional gene expression. The platform addresses several limitations associated with conventional mRNA therapeutics, including high dosing requirements and the inability to selectively express therapeutic genes based on cellular conditions.

The platform utilizes a structuring sequence that facilitates the folding of mRNA molecules into compact nanostructures. These nanostructures may encapsulate functional elements such as the 5′-untranslated region (5′-UTR) within the folded architecture, thereby preventing premature translation until specific cellular conditions are met. The conditional expression mechanism may be triggered by the presence of target cell nucleic acid molecules that are associated with disease states, viral infections, or other cellular markers.

The folded nanostructures may provide enhanced stability and protection for the nucleic acid cargo during delivery and storage. The compact folding may improve encapsulation efficiency in lipid nanoparticles and other delivery vehicles, potentially reducing the dosage requirements compared to conventional mRNA therapeutics. The structuring sequence may incorporate various structural motifs, including double crossover motifs, paranemic crossover motifs, and kissing loops, which contribute to the formation and maintenance of the folded architecture.

The platform may enable stoichiometric co-delivery of multiple therapeutic mRNAs through the formation of multimeric structures. These multimeric assemblies may be formed through intermolecular interactions between kissing loops on different nanostructures, allowing for precise control over the relative amounts of different therapeutic proteins expressed within target cells.

The conditional expression mechanism may provide tissue-specific or cell-type-specific delivery of therapeutic genes. The trigger sequences may be designed to recognize nucleic acid molecules that are present in diseased cells but absent in healthy cells, thereby minimizing off-target effects and improving the therapeutic index of the treatment. This selectivity may be particularly beneficial for applications in cancer therapy, where the expression of therapeutic genes may be limited to tumor cells containing specific oncogenes or viral sequences.

The nanostructures may be designed to fold autonomously without the need for helper nucleic acid strands, simplifying the manufacturing process and reducing the complexity of the therapeutic formulation. The intramolecular folding capabilities may be achieved through careful design of complementary sequences within the structuring strand that promote the formation of stable secondary and tertiary structures.

The nanoparticle composition may comprise a folded single-stranded nucleic acid nanostructure that includes several core functional components arranged in a specific structural configuration. The nanostructure may include a 5′-untranslated region (5′-UTR), a coding sequence, a structuring sequence, a 3′-untranslated region (3′-UTR), and a polyadenosine (poly-A) tail. These components may be integrated into a single nucleic acid molecule that undergoes intramolecular folding to form a compact nanoparticle structure.

The nucleic acid nanostructure may comprise messenger RNA (mRNA), single-stranded RNA (ssRNA), single-stranded DNA (ssDNA), or a combination thereof. In some cases, the nanostructure may be composed primarily of mRNA that retains the functional elements necessary for protein translation while incorporating additional structural features that enable controlled folding and conditional expression.

The structuring sequence may be complementary to the coding sequence and the 5′-UTR or a region of the coding sequence and the 5′-UTR. This complementarity may enable the structuring sequence to form base pairs with specific regions of the nanostructure, thereby directing the folding process and establishing the three-dimensional architecture of the assembled particle. The structuring sequence may be positioned downstream of the coding sequence and may contain multiple complementary regions that interact with different portions of the upstream nucleic acid elements.

The structuring sequence may be bound to the 5′-UTR and encapsulated within the nanostructure. This binding interaction may sequester the 5′-UTR within the folded architecture, rendering the 5′-UTR inaccessible to cellular machinery under normal conditions. The encapsulation of the 5′-UTR may occur through the formation of base pairs between complementary sequences in the 5′-UTR and corresponding regions within the structuring sequence.

The nucleic acid nanostructure may have intramolecular folding capabilities without the need for helper nucleic acid strands. The folding process may be driven by complementary base pairing interactions between different regions of the same nucleic acid molecule, allowing the nanostructure to adopt its folded conformation through self-assembly mechanisms. This autonomous folding capability may simplify the manufacturing and formulation processes by eliminating the requirement for additional oligonucleotides or cofactors.

The structuring sequence may be bound to the 5′-UTR or a region of the 5′-UTR such that the 5′-UTR cannot bind to a ribosome or be degraded by a nuclease. This protective binding may prevent premature translation initiation by blocking ribosome access to the ribosome binding site and start codon within the 5′-UTR. Additionally, the sequestration of the 5′-UTR within the folded structure may provide protection against nuclease-mediated degradation, thereby enhancing the stability of the nanostructure during storage and delivery.

The 3′-untranslated region (3′-UTR) and polyadenosine (poly-A) tail may be positioned at the 3′ end of the nanostructure, downstream of the structuring sequence. These elements may remain accessible for interaction with cellular translation machinery and may contribute to the overall stability and translation efficiency of the mRNA once the 5′-UTR is released from its sequestered state. The poly-A tail may be located at various positions within the nanostructure, including before the structuring sequence, after the structuring sequence, or at the 3′-end, depending on the specific design requirements.

The design pipeline for creating nanostructured mRNA constructs may utilize a systematic computational approach that enables the generation of folded nucleic acid nanoparticles with defined structural and functional properties. The design pipeline may begin with the selection of a target coding sequence and the subsequent development of a complementary structuring sequence that directs the folding of the mRNA molecule into a compact nanoparticle configuration.

The sequence design algorithm may analyze the target mRNA sequence to identify optimal regions for complementary base pairing interactions that will facilitate the desired folding pattern. The algorithm may consider factors such as thermodynamic stability, secondary structure formation, and accessibility of functional elements when designing the structuring sequence. The computational approach may evaluate multiple potential structuring sequences and select configurations that provide stable folding while maintaining the functional integrity of the mRNA components.

The design pipeline may incorporate computational validation and characterization steps that assess the predicted three-dimensional structure of the folded nanoparticle. These validation steps may utilize molecular modeling techniques to predict the folding behavior and structural stability of the designed nanostructure. The computational validation may include analysis of base pairing patterns, evaluation of structural motifs, and assessment of the accessibility of functional elements within the folded configuration.

The nanoparticles may be produced using in vitro transcription off of a linearized plasmid molecule encoding a nanoparticle coding sequence and downstream structuring sequence. The plasmid-based production system may enable the synthesis of full-length mRNA molecules that contain both the functional coding elements and the structural components necessary for folding. The linearized plasmid may serve as a template for RNA polymerase-mediated transcription, resulting in the production of single-stranded RNA molecules that contain the complete nanoparticle sequence.

The computational approach may enable the generation of nanoparticles with arbitrary coding sequences by adapting the structuring sequence design to accommodate different target genes. The algorithm may analyze the sequence composition and structural features of various coding sequences to develop customized structuring sequences that promote proper folding regardless of the specific gene being encoded. This flexibility may allow the platform to be applied to a wide range of therapeutic applications without requiring fundamental changes to the underlying design principles.

The design pipeline may also enable the specification of arbitrary conditions for conditional release of the 5′-UTR by incorporating trigger sequence recognition elements into the structuring sequence design. The computational approach may analyze potential trigger sequences that are associated with specific cellular states or disease conditions and design complementary regions within the structuring sequence that will respond to these triggers through strand displacement mechanisms. This capability may allow for the development of highly specific therapeutic formulations that are activated only in the presence of particular cellular markers or pathogenic nucleic acids.

The sequence design algorithm may optimize the positioning and length of complementary regions within the structuring sequence to achieve the desired balance between structural stability and trigger responsiveness. The algorithm may evaluate the thermodynamic properties of different base pairing configurations to ensure that the folded structure remains stable under physiological conditions while still being responsive to the intended trigger sequences. The computational validation may include modeling of the strand displacement kinetics to predict the efficiency and specificity of the conditional release mechanism.

The folded nucleic acid nanostructure may exhibit a complex three-dimensional architecture that demonstrates the capability of single-stranded nucleic acid molecules to adopt highly organized structural configurations through intramolecular folding mechanisms. The nanostructure may display a substantially cylindrical tube shape formed by the self-assembly of complementary sequences within the structuring strand. The cylindrical architecture may be characterized by two elongated, parallel regions with densely packed nucleic acid strands that create a compact tubular arrangement.

The substantially cylindrical tube shape may be achieved through the formation of multiple base pairing interactions between different regions of the structuring sequence and corresponding complementary sequences within the coding sequence and 5′-UTR. The parallel regions of the nanostructure may be connected by less densely packed segments that extend outward from the main cylindrical body. These extending segments may represent functional domains such as untranslated regions or other sequence elements that remain accessible for biological interactions while the core structure maintains its compact folded configuration.

The nucleic acid nanostructure may have intramolecular folding capabilities without the need for helper nucleic acid strands. The folding process may rely entirely on complementary base pairing interactions within the single nucleic acid molecule, eliminating the requirement for additional oligonucleotides or trans-acting factors to achieve the desired three-dimensional structure. This autonomous folding capability may be facilitated by the strategic placement of complementary sequences within the structuring strand that promote the formation of stable secondary and tertiary structures through Watson-Crick base pairing.

The transformation from an unfolded to a folded state may involve a reorganization of the nucleic acid molecule from an extended linear configuration to a compact three-dimensional structure. The unfolded mRNA nanostructure may be in its native, extended conformation where the nucleic acid sequence exists in a relatively open and linear arrangement. The unfolded configuration may display the backbone and base pairing regions of the mRNA molecule distributed along the length of the extended structure without the compact organization characteristic of the folded state.

The folded mRNA nanostructure may adopt the substantially cylindrical tube shape following thermal annealing or other folding conditions. The folded configuration may be significantly more compact compared to the unfolded state, with the nucleic acid strands organized into a dense, three-dimensional assembly. The transformation may be facilitated by kissing loop interactions, which may be indicated by complementary loop sequences that interact to stabilize the folded structure and maintain the tubular geometry.

The kissing loop pairs may contribute to the formation and maintenance of the substantially cylindrical tube shape by bringing distant regions of the mRNA molecule into proximity through specific base pairing interactions. These kissing loop interactions may serve as structural anchors that help to establish and maintain the overall architecture of the folded nanostructure. The 5′-UTR and poly(A) tail may be positioned at specific locations within the folded structure, with the 5′-UTR potentially sequestered within the internal cavity of the cylindrical tube.

The folded mRNA nanostructure may reveal the circular cross-section characteristic of the substantially cylindrical tube shape. The nanostructure may appear as a compact, three-dimensional assembly with nucleic acid strands forming the walls of the cylindrical structure. The front-facing perspective may demonstrate how the intramolecular folding creates a defined tubular geometry with internal space that may accommodate functional elements such as the sequestered 5′-UTR.

The structuring sequence may comprise a long hairpin along the inside edge of the cylindrical tube. This long hairpin may be embedded within the folded architecture and may serve multiple functions, including the sequestration of the 5′-UTR and the maintenance of the overall structural integrity of the nanoparticle. The hairpin structure may be formed through complementary base pairing within the structuring sequence and may be positioned along the internal surface of the cylindrical tube where the hairpin may interact with other functional elements of the nanostructure.

The long hairpin along the inside edge of the cylindrical tube may contain specific sequence elements that are complementary to regions of the 5′-UTR, enabling the sequestration of the 5′-UTR within the folded structure. The positioning of the hairpin along the inside edge may provide a protected environment for the 5′-UTR while maintaining the structural stability of the overall nanoparticle. The hairpin may also serve as a recognition site for trigger sequences that may initiate strand displacement reactions leading to the release of the sequestered 5′-UTR.

The tubular architecture may provide internal space where functional elements may be encapsulated and protected from degradation or unwanted interactions with cellular machinery. The cylindrical geometry may be maintained through multiple intramolecular interactions, including the long hairpin structure and other complementary base pairing regions within the structuring sequence. The compact folding may result in a nanostructure that exhibits enhanced stability and improved handling characteristics compared to unfolded nucleic acid molecules.

The structuring sequence may comprise one or more double crossover motifs, paranemic crossover motifs, kissing loops, or combinations thereof that contribute to the formation and stabilization of the folded nucleic acid nanostructure. These structural motifs may provide the molecular framework that enables the single-stranded nucleic acid molecule to adopt and maintain a compact three-dimensional configuration through specific intermolecular and intramolecular interactions.

Double crossover (DX) motifs may be incorporated into the structuring sequence to create stable junction points where two double-stranded regions cross over each other in a controlled manner. The DX motifs may facilitate the formation of rigid structural elements within the folded nanostructure by establishing fixed geometric relationships between different segments of the nucleic acid molecule. These crossover points may contribute to the overall structural integrity of the nanoparticle by preventing unwanted flexibility or deformation of the folded architecture.

Paranemic crossover (PX) motifs may provide an alternative crossover configuration that enables the formation of parallel double-stranded regions with controlled spacing and orientation. The PX motifs may differ from DX motifs in their geometric arrangement and may offer distinct advantages for certain structural configurations. The incorporation of PX motifs into the structuring sequence may enable the formation of more complex three-dimensional architectures while maintaining the stability and functionality of the folded nanostructure.

The structuring sequence may comprise one to nine DX or PX motifs, with different embodiments differing in the number of crossover motifs, the relative spacing of crossover motifs to each other, and the location of crossover motifs relative to the ribosome-binding sequence and start codon. The number of crossover motifs may be selected based on the desired structural properties and the length of the coding sequence being incorporated into the nanostructure. Embodiments with fewer crossover motifs may provide simpler folding patterns that are suitable for shorter coding sequences, while embodiments with more crossover motifs may enable the formation of more complex structures that can accommodate longer therapeutic genes.

The relative spacing of crossover motifs to each other may be optimized to achieve the desired balance between structural stability and functional accessibility. The spacing between adjacent crossover motifs may influence the overall geometry of the folded structure and may affect the positioning of functional elements such as the 5′-UTR and coding sequence within the three-dimensional architecture. Different spacing configurations may be employed to create nanostructures with varying degrees of compactness and different internal cavity dimensions.

The location of crossover motifs relative to the ribosome-binding sequence (RBS) and start codon may be categorized into different positional arrangements, including RBS-proximal, mid-CDS, and 3′-proximal configurations. RBS-proximal crossover motifs may be positioned near the ribosome-binding sequence and may influence the accessibility of the translation initiation machinery to the 5′-UTR. Mid-CDS crossover motifs may be located within the central regions of the coding sequence and may contribute to the overall structural organization without directly affecting translation initiation. The 3′-proximal crossover motifs may be positioned toward the 3′ end of the coding sequence and may influence the folding of the downstream structural elements.

The kissing loops may serve as intermolecular interaction sites that enable the formation of stable associations between different regions of the folded nanostructure or between multiple nanostructures. The kissing loops may be formed by complementary loop sequences that can recognize and bind to each other through specific base pairing interactions. As shown in the transition from the unfolded state to the folded state, the kissing loops may play a role in directing the folding process and maintaining the structural integrity of the assembled nanoparticle.

A composition may comprise a plurality of folded nucleic acid nanostructures stacked in a multimer via one or more kissing loops. The multimeric assemblies may be formed through intermolecular kissing loop interactions between different nanostructures, enabling the creation of larger structural complexes with enhanced functional capabilities. The stacking of multiple nanostructures may provide opportunities for stoichiometric co-delivery of different therapeutic genes or the creation of protected compartments between adjacent nanostructures.

The kissing loops may enable the formation of multimeric structures by providing specific recognition sites that allow different nanostructures to associate with each other in a controlled manner. The complementary sequences within the kissing loops may ensure that the multimeric assembly occurs with the desired stoichiometry and structural organization. The multimeric configuration may enhance the stability of the individual nanostructures while providing additional functional capabilities that are not available in monomeric forms.

The combination of double crossover motifs, paranemic crossover motifs, and kissing loops within the structuring sequence may provide a versatile toolkit for creating nanostructures with diverse structural and functional properties. The specific combination of these motifs may be tailored to the requirements of different therapeutic applications, enabling the development of customized nanoparticles that are optimized for specific delivery and expression requirements. The modular nature of these structural elements may facilitate the systematic design and optimization of nanostructures for various biomedical applications.

The kissing loop hairpins may serve as intermolecular recognition elements that enable the formation of multimeric structures through specific complementary loop sequences. The kissing loop hairpins may be incorporated into the structuring sequence of individual folded nucleic acid nanostructures, providing sites for intermolecular association between different nanoparticles. Each kissing loop hairpin may contain a loop sequence that is complementary to a corresponding loop sequence on another nanostructure, enabling the formation of stable intermolecular base pairing interactions.

The complementary loop sequences within the kissing loop hairpins may facilitate the recognition and binding between different folded nucleic acid nanostructures through Watson-Crick base pairing. The specificity of these interactions may be determined by the sequence composition of the loop regions, allowing for the selective association of nanostructures that contain matching complementary sequences. The kissing loop interactions may occur between loop regions that protrude from the main body of the folded nanostructures, enabling intermolecular contact while maintaining the structural integrity of the individual particles.

The kissing loop pairs may contribute to the overall structural organization of the folded nanostructures and may provide additional stabilization through intermolecular interactions. The kissing loops may be positioned at specific locations within the folded architecture to enable controlled stacking arrangements between multiple nanostructures. The positioning of these kissing loop elements may be designed to promote the formation of ordered multimeric assemblies with defined stoichiometric relationships.

Compositions may comprise a plurality of folded nucleic acid nanostructures stacked in a multimer via one or more kissing loops. The multimeric structures may be formed through the association of individual nanoparticles that contain complementary kissing loop hairpins, enabling the creation of larger assemblies with enhanced functional capabilities. The stacking arrangement may be achieved through the formation of intermolecular base pairs between the complementary loop sequences of adjacent nanostructures, creating stable multimeric complexes that maintain the structural integrity of the individual components.

The stacking of multiple folded nucleic acid nanostructures via kissing loops may enable stoichiometric co-delivery of structured, therapeutic mRNAs. The multimeric configuration may allow for the precise control of the relative amounts of different therapeutic proteins that are expressed within target cells by incorporating nanostructures encoding different genes into the same multimeric assembly. The stoichiometric relationships between different therapeutic mRNAs may be determined by the number and arrangement of nanostructures within the multimeric complex, providing a mechanism for achieving defined ratios of therapeutic protein expression.

The multimeric structures formed through kissing loop interactions may create protected areas between adjacent nanostructures that can conceal functional elements from cellular machinery or environmental factors. These protected areas may be formed in the interface regions between stacked nanostructures, where the kissing loop interactions bring the surfaces of adjacent particles into close proximity. The protected areas may provide additional shielding for sensitive functional elements such as untranslated regions, regulatory sequences, or other nucleic acid components that may benefit from enhanced protection during storage, delivery, or cellular uptake.

The protected areas between nanostructures in the multimeric assemblies may be utilized to conceal one or more UTRs and protect these functional elements from degradation prior to transfection. The positioning of UTRs within the protected interface regions may provide enhanced stability compared to UTRs that are exposed on the surface of individual nanostructures. The protection afforded by the multimeric configuration may extend the shelf life of the therapeutic formulation and may improve the functional integrity of the nucleic acid components during the delivery process.

The kissing loop-mediated stacking may enable the formation of multimeric structures with conditional unfolding capabilities, where the release of functional elements from one nanostructure may influence the structural stability or functional accessibility of adjacent nanostructures within the multimeric assembly. The conditional unfolding mechanism may be triggered by the presence of specific cellular factors or trigger sequences that initiate strand displacement reactions, leading to the sequential or coordinated release of functional elements from multiple nanostructures within the multimeric complex.

The stoichiometric co-delivery capability provided by the multimeric structures may be particularly beneficial for therapeutic applications that require the coordinated expression of multiple genes, such as combination therapies, multi-subunit protein complexes, or treatment regimens that involve both therapeutic genes and adjuvant factors. The ability to control the relative expression levels of different therapeutic proteins through the multimeric stacking arrangement may provide enhanced therapeutic efficacy compared to separate delivery of individual components.

The 5′-UTR protection and encapsulation mechanism may rely on the specific binding interaction between the structuring sequence and the 5′-UTR to create a sequestered state that prevents premature translation initiation. The structuring sequence may be bound to the 5′-UTR through complementary base pairing interactions that render the 5′-UTR inaccessible to cellular ribosomes and translation machinery. This binding interaction may effectively mask the ribosome binding site and start codon within the 5′-UTR, preventing the initiation of protein synthesis until the appropriate cellular conditions trigger the release mechanism.

The binding of the structuring sequence to the 5′-UTR may create a protected environment that shields the 5′-UTR from nuclease-mediated degradation. Nucleases may be unable to access the sequestered 5′-UTR due to the formation of stable secondary structures and the physical occlusion created by the base pairing interactions with the structuring sequence. This protection mechanism may enhance the stability of the folded nucleic acid nanostructure during storage, delivery, and cellular uptake by preventing the degradation of functional elements that are necessary for subsequent translation.

The conditional release mechanism may be initiated by the addition of a trigger strand that competes with the 5′-UTR for binding to the structuring sequence. The trigger strand may contain sequences that are complementary to regions of the structuring sequence that are currently bound to the 5′-UTR. The introduction of the trigger strand may initiate a strand displacement reaction that progressively displaces the 5′-UTR from its sequestered position within the folded nanostructure, thereby making the 5′-UTR accessible for ribosome binding and translation initiation.

The strand displacement mechanism may occur through a toehold-mediated process where the trigger strand initially binds to an exposed region of the structuring sequence and then progressively displaces the 5′-UTR through branch migration. The thermodynamic favorability of the trigger strand binding may drive the displacement reaction, with the trigger strand forming more stable base pairing interactions with the structuring sequence than the original 5′-UTR binding configuration. The displacement process may result in the release of the 5′-UTR from its protected state and the formation of a new stable complex between the trigger strand and the structuring sequence.

The folded mRNA nanostructure in its inactive state may demonstrate the encapsulation of the 5′-UTR within the structural framework of the nanoparticle. The 5′-UTR may be shown in a bound state where the 5′-UTR is sequestered within the nanostructure through base pairing interactions with complementary regions of the structuring sequence. The bound 5′-UTR may be positioned within the internal architecture of the folded nanostructure, where the 5′-UTR is protected from external cellular factors while remaining poised for conditional release upon trigger activation.

The structuring sequence may comprise a long hairpin along the inside edge of the cylindrical tube that serves as the primary binding site for the 5′-UTR sequestration. The long hairpin may be formed through intramolecular base pairing within the structuring sequence and may be positioned along the internal surface of the cylindrical nanostructure. The hairpin structure may contain a loop region or stem region that is complementary to specific sequences within the 5′-UTR, enabling the formation of stable intermolecular base pairs that maintain the sequestered state of the 5′-UTR.

The long hairpin along the inside edge of the cylindrical tube may be embedded within the folded architecture of the nanostructure, where the hairpin may interact directly with the 5′-UTR to maintain the inactive state of the mRNA molecule. The positioning of the long hairpin along the inside edge may provide optimal accessibility for 5′-UTR binding while maintaining the structural integrity of the overall cylindrical geometry. The hairpin may extend into the internal cavity of the cylindrical tube, creating a binding pocket or recognition site that accommodates the 5′-UTR in its sequestered configuration.

The length of the long hairpin may affect the binding affinity and release kinetics of the 5′-UTR sequestration mechanism. Longer hairpins may provide more extensive base pairing interactions with the 5′-UTR, resulting in stronger binding and potentially requiring more stringent trigger conditions for release. Shorter hairpins may enable more rapid release kinetics but may provide less stable sequestration of the 5′-UTR. The optimal hairpin length may be determined based on the specific requirements for binding stability and trigger responsiveness for different therapeutic applications.

The factors affecting binding and release of the 5′-UTR may include the thermodynamic stability of the base pairing interactions between the structuring sequence and the 5′-UTR, the accessibility of the binding sites within the folded nanostructure, and the presence of competing nucleic acid sequences that may interfere with the sequestration mechanism. The binding affinity may be influenced by the length and sequence composition of the complementary regions, with longer and more perfectly matched sequences generally providing stronger binding interactions.

The release of the sequestered 5′-UTR may depend on the presence of trigger sequences that can compete effectively with the existing base pairing interactions. The trigger sequences may need to have higher binding affinity for the structuring sequence than the 5′-UTR in order to drive the strand displacement reaction. The concentration of trigger sequences within the target cell may also influence the efficiency and kinetics of the release mechanism, with higher concentrations generally promoting more rapid and complete displacement of the sequestered 5′-UTR.

The encapsulation of the 5′-UTR within the nanostructure may be maintained through multiple stabilizing interactions, including the primary base pairing with the long hairpin and additional secondary interactions with other regions of the structuring sequence. The encapsulated state may be further stabilized by the overall three-dimensional architecture of the folded nanostructure, which may provide physical constraints that help to maintain the sequestered position of the 5′-UTR. The combination of these stabilizing factors may ensure that the 5′-UTR remains in its inactive state until the appropriate trigger conditions are encountered within the target cell.

The conditional release mechanism may be initiated through a strand displacement process that enables the transition from an inactive OFF state to an active ON state in response to specific cellular conditions. The strand displacement mechanism may rely on the competitive binding of a target cell nucleic acid molecule to the structuring sequence, which may displace the sequestered 5′-UTR and enable translation initiation. The target cell nucleic acid molecule may serve as a trigger strand that recognizes and binds to complementary sequences within the structuring sequence, thereby initiating the release process.

The target cell nucleic acid molecule may be present within specific cell types or under particular cellular conditions, such as viral infection, disease states, or the presence of specific biomarkers. The target cell nucleic acid molecule may comprise sequences that are associated with pathogenic organisms, oncogenes, viral genomes, or other disease-related nucleic acids that are present in target cells but absent in healthy cells. The specificity of the target cell nucleic acid molecule recognition may enable selective activation of the therapeutic gene expression only in cells that contain the appropriate trigger sequences.

The method for selectively expressing a coding sequence in a target cell may comprise delivering to a target cell a nanoparticle composition comprising the folded single-stranded nucleic acid nanostructure. Upon delivering the nanoparticle composition to the target cell, a target cell nucleic acid molecule may bind the structuring sequence and displace the 5′-UTR, thereby releasing the 5′-UTR for translation and expression of the coding sequence. The delivery process may involve cellular uptake of the nanoparticle composition through endocytosis, membrane fusion, or other cellular internalization mechanisms.

The binding of the target cell nucleic acid molecule to the structuring sequence may occur through complementary base pairing interactions that are thermodynamically favored over the existing base pairing between the structuring sequence and the 5′-UTR. The target cell nucleic acid molecule may contain sequences that exhibit higher binding affinity for the structuring sequence compared to the sequestered 5′-UTR, thereby driving the displacement reaction in the forward direction. The competitive binding process may be facilitated by the accessibility of binding sites within the structuring sequence and the concentration of the target cell nucleic acid molecule within the cellular environment.

The OFF state of the mRNA nanoparticle may be characterized by the sequestration of the 5′-UTR within the folded structure, where the 5′-UTR remains bound to the structuring sequence and inaccessible to cellular translation machinery. In the OFF state, the 5′-UTR may be positioned within the internal architecture of the nanostructure through base pairing interactions with complementary regions of the structuring sequence. The sequestered 5′-UTR may be unable to interact with ribosomes or other translation initiation factors, effectively preventing the expression of the coding sequence until the appropriate trigger conditions are encountered.

The OFF state may be maintained through stable base pairing interactions between the 5′-UTR and the structuring sequence, which may include interactions with the long hairpin along the inside edge of the cylindrical tube. The structural stability of the OFF state may be sufficient to prevent spontaneous release of the 5′-UTR under normal cellular conditions, ensuring that gene expression remains suppressed until the specific target cell nucleic acid molecule is present. The OFF state configuration may also provide protection for the 5′-UTR against nuclease degradation and other cellular factors that might otherwise compromise the functional integrity of the nanostructure.

The ON state of the mRNA nanoparticle may be achieved following the encounter with a trigger strand that initiates the strand displacement mechanism. In the ON state, the 5′-UTR may be released from its sequestered position and may become accessible for ribosome binding and translation initiation. The transition to the ON state may result in the formation of a new stable complex between the target cell nucleic acid molecule and the structuring sequence, while the displaced 5′-UTR may adopt an extended conformation that enables interaction with cellular translation machinery.

The strand displacement process leading to the ON state may occur through a toehold-mediated mechanism where the target cell nucleic acid molecule initially binds to an exposed region of the structuring sequence and subsequently displaces the 5′-UTR through branch migration. The displacement reaction may proceed in a stepwise manner, with the target cell nucleic acid molecule progressively forming base pairs with the structuring sequence while simultaneously disrupting the existing base pairs between the structuring sequence and the 5′-UTR. The kinetics of the strand displacement reaction may be influenced by the sequence composition of the target cell nucleic acid molecule, the binding affinity differences between the competing sequences, and the structural accessibility of the binding sites.

The trigger strand may become bound to a hairpin structure within the folded nanostructure, forming a stable complex that maintains the ON state configuration. The binding of the trigger strand to the hairpin may result in the formation of a double-stranded region that is thermodynamically stable and resistant to spontaneous dissociation. The trigger strand bound to the hairpin may serve as a molecular switch that locks the nanostructure in the active configuration, ensuring sustained gene expression once the activation process has been initiated.

The released 5′-UTR in the ON state may be available for translation and expression of the coding sequence through normal cellular translation mechanisms. The unbound 5′-UTR may interact with ribosomal subunits, translation initiation factors, and other components of the protein synthesis machinery to enable the production of the encoded therapeutic protein. The accessibility of the 5′-UTR in the ON state may restore the normal translation capabilities of the mRNA molecule, allowing for efficient protein expression within the target cell.

The strand displacement mechanism may be illustrated through the transition between different structural states of the mRNA nanoparticle. The mechanism may demonstrate how the presence of specific trigger sequences within target cells can selectively activate gene expression while maintaining the inactive state in cells that lack the appropriate trigger molecules. The selectivity of the activation mechanism may be determined by the sequence specificity of the target cell nucleic acid molecule recognition and the thermodynamic favorability of the strand displacement reaction.

The method for selectively expressing a coding sequence in a target cell may provide tissue-specific or cell-type-specific activation of therapeutic gene expression based on the presence of characteristic nucleic acid molecules within different cellular environments. The target cell nucleic acid molecule may be associated with a virus or a disease, enabling the selective activation of therapeutic genes in infected or diseased cells while avoiding activation in healthy cells. The disease-associated target cell nucleic acid molecules may include viral RNA sequences, oncogene transcripts, pathogen-derived nucleic acids, or other molecular markers that are characteristic of specific disease states.

The strand displacement mechanism may enable the development of precision therapeutics that respond to specific cellular conditions or disease markers. The ability to conditionally activate gene expression based on the presence of target cell nucleic acid molecules may improve the therapeutic index of nucleic acid-based treatments by reducing off-target effects and enhancing the specificity of therapeutic intervention. The conditional activation mechanism may be particularly beneficial for applications in cancer therapy, antiviral treatments, and other therapeutic areas where selective targeting of diseased cells is desirable.

The efficiency of the strand displacement reaction may be influenced by the concentration of the target cell nucleic acid molecule within the target cell, the binding kinetics of the competitive interactions, and the structural accessibility of the binding sites within the folded nanostructure. Higher concentrations of the target cell nucleic acid molecule may promote more rapid and complete activation of the gene expression mechanism, while lower concentrations may result in partial or delayed activation. The design of the structuring sequence and the selection of appropriate target cell nucleic acid molecule recognition sequences may be optimized to achieve the desired sensitivity and specificity for different therapeutic applications.

The coding sequence incorporated into the folded single-stranded nucleic acid nanostructure may comprise various therapeutic and immunogenic nucleic acid sequences that enable the nanoparticle composition to function as a targeted therapeutic or vaccine platform. The coding sequence may comprise a viral nucleic acid sequence, a bacterial nucleic acid sequence, or a parasitic nucleic acid sequence, depending on the specific therapeutic application and target pathogen or disease state. The selection of the coding sequence may be tailored to the intended therapeutic outcome, whether for vaccine development, therapeutic protein expression, or targeted treatment of specific diseases.

The viral nucleic acid sequence may be derived from SARS CoV2, influenza virus, or HPV, providing the nanoparticle composition with the capability to elicit immune responses against these clinically relevant pathogens. The viral nucleic acid sequences may encode antigenic proteins or protein fragments that serve as targets for immune recognition and response generation. The incorporation of viral nucleic acid sequences into the nanostructure may enable the development of vaccines that provide protection against viral infections through the induction of both humoral and cellular immune responses.

The coding sequence may comprise whole genes that encode complete functional proteins with therapeutic or immunogenic properties. The whole genes may include sequences encoding the SARS CoV2 spike protein, which serves as a primary target for vaccine development against COVID-19. The SARS CoV2 spike protein coding sequence may enable the nanoparticle composition to function as a vaccine that induces protective immunity against SARS CoV2 infection through the expression of the spike protein antigen within target cells.

The incorporation of neoantigen sequences into the coding sequence may enable the development of personalized cancer vaccines that target tumor-specific mutations. Neoantigens may represent ideal targets for cancer immunotherapy due to their tumor-specific expression and lack of central immune tolerance. The neoantigen mRNA may be designed based on the specific mutational profile of individual tumors, enabling the development of personalized therapeutic approaches that target the unique antigenic landscape of each patient's cancer.

The coding sequence may be designed to accommodate various therapeutic applications through the modular incorporation of different functional elements. The flexibility of the nanostructure platform may enable the rapid adaptation of the coding sequence to address emerging therapeutic needs or to incorporate newly identified antigenic targets. The modular design approach may facilitate the development of combination therapies that incorporate multiple therapeutic genes or immunostimulatory factors within a single nanoparticle formulation.

The combination of immunostimulatory coding sequences, reporter genes, and additional sequence elements may provide a comprehensive platform for therapeutic development and research applications. The modular nature of these sequence components may enable the customization of nanostructure formulations for specific therapeutic applications while maintaining the ability to monitor and quantify the performance of the delivery system. The incorporation of multiple functional elements within a single nanostructure may provide enhanced therapeutic efficacy compared to formulations that deliver individual components separately.

The 5′-untranslated region (5′-UTR) and 3′-untranslated region (3′-UTR) within the folded single-stranded nucleic acid nanostructure may be configured in various arrangements to optimize translation efficiency and provide enhanced functional capabilities. The UTR configurations may be selected based on the specific requirements for translation initiation, mRNA stability, and cellular localization of the therapeutic proteins encoded by the nanostructure. The selection of appropriate UTR sequences may influence the overall performance of the nanoparticle composition by affecting both the efficiency of protein expression and the cellular response to the delivered nucleic acid.

The UTR configurations may be customized for different therapeutic applications based on the specific requirements for protein expression levels, expression kinetics, and cellular targeting. Different combinations of murine beta-globin-derived UTRs and RHA recruitment sequences may be evaluated to identify optimal configurations for specific therapeutic proteins or target cell types. The modular nature of the UTR design may enable rapid optimization of nanostructure performance for various therapeutic applications without requiring fundamental changes to the overall folding and conditional expression mechanisms.

The polyadenosine (poly-A) tail within the folded single-stranded nucleic acid nanostructure may be positioned at various locations to optimize translation initiation and mRNA circularization mechanisms. The positioning of the poly-A tail may significantly influence the efficiency of protein synthesis by affecting the recruitment and organization of translation initiation factors and RNA-binding proteins that facilitate ribosome assembly and mRNA processing. The strategic placement of the poly-A tail may enable fine-tuning of the translation characteristics of the nanostructure to achieve desired levels of therapeutic protein expression.

The poly-A tail may be located before the structuring sequence, positioning this regulatory element upstream of the folding domain within the linear organization of the nanostructure. The placement of the poly-A tail before the structuring sequence may enable the poly-A tail to remain accessible for interaction with cellular factors even when the nanostructure adopts its folded configuration. This positioning may facilitate the recruitment of poly-A-binding protein (PABP) and associated translation factors prior to or during the folding process, potentially enhancing the overall translation competency of the nanostructure.

The nucleic acid nanostructure may further comprise one or more DNA or RNA staple strands that provide additional structural support to enhance the stability and structural integrity of the folded configuration. The staple strands may function as auxiliary structural elements that complement the intramolecular folding capabilities of the primary nucleic acid molecule by providing additional base pairing interactions and structural constraints that stabilize the three-dimensional architecture of the nanoparticle.

The DNA staple strands may comprise short oligonucleotide sequences that are complementary to specific regions of the folded nucleic acid nanostructure. These DNA staple strands may form Watson-Crick base pairs with designated binding sites within the structuring sequence or other regions of the nanostructure, thereby providing additional structural reinforcement through intermolecular interactions. The DNA staple strands may be designed to bind to accessible regions of the folded nanostructure without interfering with the functional elements such as the coding sequence, UTRs, or the conditional release mechanism.

The RNA staple strands may provide structural support through similar base pairing mechanisms while offering enhanced compatibility with the RNA-based components of the nanostructure. The RNA staple strands may form more stable interactions with the RNA nanostructure compared to DNA staple strands due to the similar chemical properties and structural characteristics of RNA molecules. The RNA staple strands may be synthesized using standard RNA synthesis techniques and may be incorporated into the nanostructure formulation as separate oligonucleotide components.

The staple strands may be designed to bind to specific regions of the structuring sequence that are accessible in the folded configuration but do not interfere with the base pairing interactions that maintain the sequestered state of the 5′-UTR. The positioning of staple strand binding sites may be selected to provide structural reinforcement without compromising the conditional release mechanism or the functional integrity of the coding sequence and regulatory elements. The staple strands may bind to loop regions, single-stranded overhangs, or other accessible sites within the folded architecture.

The length of the staple strands may be optimized to provide sufficient binding affinity for stable association with the nanostructure while avoiding excessive length that might interfere with cellular uptake or processing. Shorter staple strands may provide adequate structural support while minimizing the overall complexity of the nanoparticle formulation. Longer staple strands may provide enhanced binding stability but may require more careful design to avoid unwanted interactions with cellular machinery or off-target binding to endogenous nucleic acids.

The concentration of staple strands relative to the primary nanostructure may be optimized to achieve complete binding occupancy at the designated binding sites while avoiding excess staple strands that might interfere with cellular uptake or processing. The stoichiometric ratio between the nanostructure and staple strands may be determined through empirical optimization based on binding affinity measurements and structural characterization studies. The optimal stoichiometry may vary depending on the specific design of the nanostructure and the number and accessibility of staple strand binding sites.

The staple strands may be chemically modified to enhance their stability, binding affinity, or cellular compatibility. Chemical modifications may include phosphorothioate linkages to provide nuclease resistance, 2′-O-methyl modifications to enhance binding affinity, or other modifications that improve the performance characteristics of the staple strands. The selection of appropriate chemical modifications may be based on the specific requirements for stability, functionality, and biocompatibility in the intended therapeutic application.

The plasmid molecule may be designed to contain the nanoparticle coding sequence positioned upstream of the structuring sequence, with appropriate regulatory elements such as a T7 promoter sequence that enables efficient RNA polymerase-mediated transcription. The coding sequence within the plasmid may comprise any of the therapeutic genes, reporter genes, or immunogenic sequences described herein, while the downstream structuring sequence may contain the complementary regions necessary for intramolecular folding and 5′-UTR sequestration. The plasmid design may incorporate standard molecular cloning techniques to ensure proper orientation and spacing of the functional elements.

The linearization of the plasmid molecule may be accomplished through restriction enzyme digestion at a specific cleavage site positioned downstream of the structuring sequence. The linearization process may create a defined 3′-end for the transcription template, ensuring that the resulting RNA transcripts contain the complete nanoparticle sequence without additional vector-derived sequences. The restriction enzyme cleavage site may be selected to provide a clean cut that does not interfere with the structural or functional elements of the nanoparticle sequence.

The linearized plasmid template may be purified through ethanol precipitation or other standard nucleic acid purification methods to remove restriction enzymes, buffer components, and other contaminants that might interfere with the subsequent transcription reaction. The ethanol precipitation process may concentrate the linearized template while removing salts and proteins that could inhibit RNA polymerase activity. The purified linearized template may be resuspended in appropriate buffer conditions for use in the in vitro transcription reaction.

The storage and handling of unfolded mRNA nanostructures may require specific conditions to maintain RNA integrity and folding potential. The RNA preparations may be stored at low temperatures to minimize degradation, and may be supplemented with RNase inhibitors or other stabilizing agents to prevent enzymatic degradation. The storage conditions may be optimized to maintain the functional integrity of the nanostructures while preserving their ability to undergo subsequent folding processes.

The production method may be adapted for the synthesis of nanostructures containing different coding sequences through the use of modular cloning approaches that enable rapid exchange of coding sequence elements within the plasmid template. The modular design may facilitate the production of nanostructure libraries containing various therapeutic genes while maintaining consistent structuring sequence elements. The flexibility of the production system may enable the rapid generation of customized nanostructures for different therapeutic applications.

The thermal annealing process may be utilized to transform the unfolded mRNA nanostructures into their folded three-dimensional configurations through controlled temperature manipulation in appropriate buffer systems. The thermal annealing method may provide a systematic approach for achieving the desired folded state by enabling the formation of stable intramolecular base pairing interactions within the structuring sequence while maintaining the functional integrity of all sequence components.

The unfolded mRNA nanostructures may be diluted to a concentration between 10-100 ng/μL in buffer solutions that provide optimal conditions for the folding process. The dilution step may reduce intermolecular interactions between different nanostructure molecules while promoting intramolecular folding within individual molecules. The concentration range may be selected to balance the efficiency of folding with the practical requirements for downstream applications, with higher concentrations potentially providing more efficient folding while lower concentrations may reduce the risk of intermolecular aggregation.

The method for selectively expressing a coding sequence in a target cell may provide a targeted therapeutic approach that enables conditional gene expression based on the presence of specific cellular markers or disease-associated nucleic acid molecules. The method may comprise delivering to a target cell a nanoparticle composition comprising the folded single-stranded nucleic acid nanostructure described herein, which contains all the functional elements necessary for conditional expression including the 5′-untranslated region, coding sequence, structuring sequence, 3′-UTR, and polyadenosine tail in their sequestered configuration.

The delivery of the nanoparticle composition to target cells may be accomplished through various administration routes and cellular uptake mechanisms that enable the nanostructures to reach their intended cellular destinations. The nanoparticle composition may be formulated with appropriate delivery vehicles, such as lipid nanoparticles, liposomes, or other carrier systems that facilitate cellular internalization while protecting the nucleic acid cargo during transport. The delivery process may involve systemic administration, local injection, or targeted delivery approaches that concentrate the nanoparticles in specific tissues or cell populations.

Upon cellular uptake, the folded nanostructures may encounter the intracellular environment where target cell nucleic acid molecules may be present at concentrations sufficient to initiate the conditional expression mechanism. The target cell nucleic acid molecule may represent endogenous RNA or DNA sequences that are characteristic of specific cellular states, disease conditions, or pathogenic infections. The presence of these target cell nucleic acid molecules within the cellular environment may serve as molecular indicators that identify cells requiring therapeutic intervention.

The target cell nucleic acid molecule may bind the structuring sequence through complementary base pairing interactions that compete with the existing base pairing between the structuring sequence and the sequestered 5′-UTR. The binding affinity of the target cell nucleic acid molecule for the structuring sequence may be higher than the affinity of the 5′-UTR for the same binding sites, thereby driving the displacement reaction in the forward direction. The competitive binding process may be facilitated by the accessibility of binding sites within the structuring sequence and the local concentration of the target cell nucleic acid molecule within the cellular environment.

The displacement of the 5′-UTR through the competitive binding mechanism may occur through a strand displacement process that progressively releases the sequestered 5′-UTR from its bound state within the folded nanostructure. The strand displacement reaction may proceed through a toehold-mediated mechanism where the target cell nucleic acid molecule initially binds to accessible regions of the structuring sequence and subsequently displaces the 5′-UTR through branch migration. The kinetics of the displacement reaction may be influenced by the binding affinities of the competing sequences, the local concentrations of the target cell nucleic acid molecule, and the structural accessibility of the binding sites.

The conditional expression mechanism may be illustrated through the comparison of different experimental conditions that demonstrate the functional expression of reporter genes from the folded nanostructures. The brightfield and fluorescence microscopy images may show the expression of enhanced green fluorescent protein from various nanostructure formulations, including lipofectamine-only negative controls, unfolded eGFP-OG nanostructures, thermally annealed eGFP-OG nanostructures, and positive control eGFP mRNA. The microscopy results may demonstrate that both unfolded and folded nanostructures are capable of supporting protein expression, indicating that the folding process does not prevent translation once the 5′-UTR becomes accessible.

The release of the 5′-UTR through the strand displacement mechanism may restore the accessibility of this regulatory element for interaction with cellular translation machinery, thereby enabling translation initiation and expression of the coding sequence. The released 5′-UTR may adopt an extended conformation that allows ribosome binding and scanning to the start codon, facilitating the normal translation process. The transition from the sequestered state to the accessible state may represent the molecular switch that converts the nanostructure from an inactive configuration to an active expression system.

The target cell nucleic acid molecule may be associated with a virus, enabling selective activation of therapeutic gene expression in virus-infected cells while maintaining the inactive state in uninfected cells. Viral nucleic acid molecules may include viral RNA genomes, viral mRNA transcripts, or viral DNA sequences that are present at elevated concentrations within infected cells. The viral nucleic acid molecules may serve as specific molecular signatures that identify cells harboring viral infections and may trigger the conditional expression of antiviral therapeutic genes or immune-stimulating factors.

The target cell nucleic acid molecule may be associated with a disease, providing a mechanism for disease-specific activation of therapeutic gene expression. Disease-associated nucleic acid molecules may include aberrantly expressed genes, mutated sequences, or pathological RNA species that are characteristic of specific disease states. The disease-associated target cell nucleic acid molecules may enable the selective treatment of diseased cells while avoiding therapeutic intervention in healthy cells that lack these molecular markers.

The target cell nucleic acid molecule may be associated with a cancer, enabling tumor-specific activation of therapeutic gene expression through recognition of cancer-associated nucleic acid sequences. Cancer-associated target cell nucleic acid molecules may include oncogene transcripts, tumor suppressor gene mutations, fusion gene products, or other nucleic acid species that are characteristic of malignant transformation. The cancer-specific activation mechanism may enable the selective expression of cytotoxic genes, immune-stimulating factors, or tumor suppressor proteins within cancer cells while sparing normal cells.

The selectivity of the conditional expression mechanism may be determined by the sequence specificity of the target cell nucleic acid molecule recognition and the thermodynamic favorability of the competitive binding interactions. The design of the structuring sequence may incorporate specific recognition elements that exhibit high binding affinity for the intended target cell nucleic acid molecules while maintaining low affinity for non-target sequences that may be present in healthy cells. The specificity of the recognition mechanism may be enhanced through the optimization of binding site sequences, the incorporation of multiple recognition elements, or the use of cooperative binding mechanisms that require the simultaneous presence of multiple target sequences.

The method for selectively expressing a coding sequence in a target cell may provide enhanced therapeutic specificity compared to conventional gene therapy approaches by limiting therapeutic gene expression to cells that contain appropriate molecular triggers. The conditional expression mechanism may reduce off-target effects and may improve the therapeutic index by concentrating therapeutic activity within diseased or infected cells while minimizing exposure of healthy cells to potentially toxic therapeutic agents. The selective activation approach may be particularly beneficial for therapeutic applications that require precise targeting of specific cell populations or disease states.

The efficiency of the selective expression method may be influenced by factors including the concentration of target cell nucleic acid molecules within the target cells, the binding kinetics of the strand displacement reaction, and the stability of the folded nanostructure configuration prior to activation. Higher concentrations of target cell nucleic acid molecules may promote more rapid and complete activation of the conditional expression mechanism, while lower concentrations may result in partial or delayed activation. The optimization of these factors may enable the development of therapeutic formulations with tailored activation characteristics for specific applications.

The method may enable the development of personalized therapeutic approaches where the target cell nucleic acid molecule recognition sequences are customized based on the specific molecular signatures present in individual patients or disease conditions. The modular design of the nanostructure platform may facilitate the rapid adaptation of the conditional expression mechanism to recognize different target sequences, enabling the development of patient-specific or disease-specific therapeutic formulations. The personalized approach may provide enhanced therapeutic efficacy by targeting the unique molecular characteristics of individual disease states or pathogenic infections.

The nanoparticle composition may comprise a plurality of therapeutic mRNAs that enable the coordinated delivery and expression of multiple therapeutic genes within target cells. The plurality of therapeutic mRNAs may be incorporated into multimeric nanostructure assemblies that provide precise control over the stoichiometric relationships between different therapeutic proteins expressed within the same cellular environment. The multi-mRNA approach may offer advantages over single-gene delivery systems by enabling the simultaneous expression of complementary therapeutic factors, regulatory proteins, and immune-stimulating molecules that work synergistically to achieve enhanced therapeutic outcomes.

The plurality of therapeutic mRNAs may be stacked in a multimer via one or more kissing loops for stoichiometric co-delivery. The kissing loop-mediated stacking mechanism may enable the formation of ordered assemblies where individual nanostructures containing different therapeutic mRNAs associate through specific intermolecular base pairing interactions between complementary loop sequences. The multimeric configuration may ensure that the different therapeutic mRNAs are delivered to target cells in defined ratios that correspond to the stoichiometric arrangement of nanostructures within the multimeric assembly.

The multimeric nanostructure assemblies may enable the precise control of expression ratios between different therapeutic factors through the stoichiometric arrangement of individual nanostructures within the multimeric complex. The expression ratios may be predetermined based on the composition of the multimeric assembly, with different ratios achievable through the incorporation of different numbers of nanostructures encoding specific therapeutic genes. The ratio control capability may enable the optimization of therapeutic combinations for specific applications and may facilitate the development of personalized therapeutic formulations.

The kissing loop-mediated assembly mechanism may provide stability to the multimeric complexes while maintaining the individual functional characteristics of each component nanostructure. The kissing loop interactions may be sufficiently stable to maintain the multimeric assembly during delivery and cellular uptake while allowing for the independent activation of individual nanostructures within the complex. The balance between assembly stability and functional independence may be optimized through the design of kissing loop sequences and the selection of appropriate base pairing strengths.

The multimeric approach may enable the development of sophisticated therapeutic programs that involve the sequential or coordinated activation of multiple therapeutic pathways within target cells. The multi-pathway activation may provide enhanced therapeutic efficacy compared to single-pathway interventions and may enable the development of more comprehensive treatment approaches for complex diseases. The coordinated activation of multiple therapeutic pathways may also provide redundancy that enhances the robustness of the therapeutic intervention and may reduce the likelihood of therapeutic resistance or failure.

The co-delivery platform may be adapted for various therapeutic applications through the modular incorporation of different therapeutic mRNAs into the multimeric assemblies. The modular design approach may enable the rapid development of customized therapeutic formulations for specific diseases or patient populations without requiring fundamental changes to the underlying delivery mechanism. The flexibility of the co-delivery platform may facilitate the translation of the technology to diverse therapeutic applications and may enable the development of personalized medicine approaches based on individual patient characteristics or disease profiles.

### Methods

Disclosed herein are methods for selectively expressing a coding sequence in a target cell. A method for selectively expressing a coding sequence in a target cell includes delivering to a target cell a nanoparticle composition described herein. Upon delivery of the nanoparticle composition to the target cell, a target cell nucleic acid molecule binds the structuring sequence and displaces the 5′-UTR, thereby releasing the 5′-UTR for translation and expression of the coding sequence.

The target cell may be a human cell. The target cell may be a tumor cell, a virus-infected cell, a microorganism-infected cell, a parasite-infected cell, an autoimmune cell, or a cell expressing a specific biomarker.

The target cell nucleic acid molecule may be associated with a virus. The target cell nucleic acid molecule may be associated with a bacterium. The target cell nucleic acid molecule may be associated with a parasite. The target cell nucleic acid molecule may be associated with a disease. The target cell nucleic acid molecule may be associated with a cancer.

A method for selectively expressing a coding sequence in a target cell is useful for a variety of purposes. A method described herein may be used for stoichiometric co-delivery of structured, therapeutic mRNAs via mRNA particle stacking with conditional unfolding. A method described herein may be used for co-delivery of transgenes. A method for co-delivery of transgenes may offer benefits in generating antigen presenting cells for cell-based therapies. A method for co-delivery of transgenes may offer benefits in co-expression of immunostimulatory receptors alongside primary antigens in a vaccine formulation. A method described herein may be used for activation and maturation of dendritic cells. A method described herein may be used for modulating Tregs into Th1-like cells. A method described herein may be used for ameliorating the immunosuppressive mechanisms of Tregs to enhance immune activation.

### Pharmaceutical Compositions

Pharmaceutical compositions described herein may comprise a nucleic acid nanostructure composition.

The nucleic acid nanostructure composition may be incorporated into pharmaceutical compositions suitable for administration to a subject (such as a patient, which may be a human or non-human). The pharmaceutical compositions may include a “therapeutically effective amount” or a “prophylactically effective amount” of the active agent (nucleic acid nanostructure composition). A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A “therapeutically effective amount” is also one in which any toxic or detrimental effects are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material, or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the nucleic acid nanostructure compositions and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the nucleic acid nanostructure composition is administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions. Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%. Suitable colorants include a colorant such as an FD&C dye. When used, the amount of colorant in a systemic or topical composition is typically about 0.005 to about 0.1%. Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%. Suitable antioxidants include butylated hydroxyanisole (“BHA”), butylated hydroxytoluene (“BHT”), and vitamin E. The amount of antioxidant(s) in a systemic or topical composition is typically about 0.1 to about 5%. Suitable preservatives include benzalkonium chloride, methyl paraben and sodium benzoate. The amount of preservative(s) in a systemic or topical composition is typically about 0.01 to about 5%. Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%. Suitable suspending agents include AVICEL RC-591 (from FMC Corporation of Philadelphia, PA) and sodium alginate. The amount of suspending agent(s) in a systemic or topical composition is typically about 1 to about 8%. Suitable surfactants include lecithin, Polysorbate 80, and sodium lauryl sulfate, and the TWEENs from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and Mccutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of actives and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active and a carrier including one or more diluents disclosed above in a capsule comprising gelatin. Granules typically comprise an active, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this disclosure. Solid compositions may be coated by conventional methods, typically with pH or time-dependent coatings, such that the nucleic acid nanostructure composition is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include the nucleic acid nanostructure composition and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed nucleic acid nanostructure composition and a carrier. The carrier of the topical composition preferably aids penetration of the nucleic acid nanostructure composition into the skin. The carrier may further include one or more optional components.

The amount of the carrier employed in conjunction with nucleic acid nanostructure composition is sufficient to provide a practical quantity of composition for administration per unit dose of the medicament. Techniques and compositions for making dosage forms useful in the methods described herein are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%. The amount of thickener(s) in a topical composition is typically about 0% to about 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%. The amount of fragrance in a topical composition is typically about 0% to about 0.5%, particularly, about 0.001% to about 0.1%. Suitable pH adjusting additives include HCl or NaOH in amounts sufficient to adjust the pH of a topical pharmaceutical composition.

### Kits

Described herein is a kit, which may be used for detecting or administering the compositions described herein. In one aspect, the kit comprises a composition described herein, a device for administering the composition, a label or instructions for use; and packaging. The kit comprises at least one component for selectively expressing a coding sequence in a target cell. For example, the kit can comprise a label or instructions for selectively expressing a coding sequence in a target cell. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The component may include at least one composition comprising a folded single-stranded nucleic acid nanostructure. The folded single-stranded nucleic acid nanostructure may include a coding sequence, a structuring sequence, a 5′-untranslated region (UTR), a 3′-UTR, or a poly-A tail. Preferably, the kit comprises all components, i.e., reagents, standards, buffers, diluents, apparata, devices, etc., which are necessary to perform the disclosed method. The kit may also include other bioactive compositions.

The kit may also include a device for administering a disclosed composition.

Described herein is an mRNA nanoparticle with a structuring strand, which allows for stacking multiple mRNA together (improving loading efficiency, thus eventually leading to lower dosage needed), and also introduces a mechanism that prevents the mRNA from expressing inside cell, unless a specific trigger sequence is already present, thus enabling precision based delivery, with promising applications also in gene therapy, vaccines, and precision therapies.

Also described herein is a gene delivery system exhibiting high stability, compactness, and structural utility. The delivery system has a built-in ON switch, which only expresses the gene only in cells that contain a specified “trigger” RNA molecule, which then enables the translation of the delivered construct. The chief purposes of this invention include RNA therapeutic and vaccine with tissue or cell-specific delivery and expressing appropriate for targeted gene therapies.

Described herein is a multi-part RNA therapeutic containing a conditionally released 5′-UTR, a coding sequence, a structuring sequence, a 3′-UTR, and a poly-A tail. One aspect is in using tandem structuring strands to selectively fold the coding sequence into a tube shape, which hides inside of it the 5′-UTR region so that it cannot be bound to ribosome. The tube shape is obtained by binding the edges of the folded squared shape with kissing loop hairpins. The 5′-UTR is hidden by binding it to the hairpin inside the mRNA nanostructure tube. Strand displacement reaction is used to release the 5′-UTR that is hidden inside the mRNA nanostructure folded into the tube shape, so that it is then accessible for ribosome binding. Furthermore, the folded mRNAs can be stacked into “sandwich” like multimers, thus allowing for precise stochiometric co-delivery of multiple different mRNAs.

A high-throughput pipeline is described for the generation of nanoparticles with arbitrary coding sequences and arbitrary conditions for conditional release of the 5′-UTR and expression of the coding sequence. Furthermore, the algorithms can be applied to various nanoparticle geometries such as larger origami particles with a higher capacity for carrying coding sequences.

This pipeline permits:
Implementation of structuring nucleic acid sequence tandem to a coding sequence on the same RNA molecule to achieve desired fold states with functional utility for folding mRNA into a tube and conditional 5′-UTR release.
Protection of single stranded 5′-UTR from degradation via encapsulation via conditional binding inside the nanostructure to a component of a structuring strand.
Using elements of the structuring strand to form intermolecular nanoparticle structures for stoichiometric delivery of e.g., gene and adjuvant proteins, or Cas9 and sgRNA.

One embodiment described herein is a nanoparticle composition comprising a folded single-stranded nucleic acid nanostructure comprising: a 5′-untranslated region (5′-UTR); a coding sequence; a structuring sequence that is complementary to the coding sequence and the 5′-UTR or a region of the coding sequence and the 5′-UTR; a 3′-untranslated region (3′-UTR); and a polyadenosine (poly-A) tail; wherein the structuring sequence is bound to the 5′-UTR and encapsulated in the nanostructure. In one aspect, the coding sequence comprises a viral nucleic acid sequence, a bacterial nucleic acid sequence, or a parasitic nucleic acid sequence. In another aspect, the viral nucleic acid sequence is derived from SARS CoV2, influenza virus, or HPV. In another aspect, the coding sequence comprises a nucleic acid encoding one or more of CD40L, CD70, TLR4, and a neoantigen. In another aspect, the coding sequence comprises a reporter gene selected from the group consisting of NanoLuc luciferase (Nluc), mCitrine, mCherry, mStrawberry, green fluorescent protein (GFP), enhanced green fluorescent protein (EGFP), yellow fluorescent protein (YFP), and red fluorescent protein (RFP). In another aspect, the nucleic acid nanostructure comprises messenger RNA (mRNA), single-stranded RNA (ssRNA), single-stranded DNA (ssDNA), or a combination thereof. In another aspect, the structuring sequence comprises one or more double crossover motifs, paranemic crossover motifs, kissing loops, or combinations thereof. In another aspect, the composition comprises a plurality of folded nucleic acid nanostructures stacked in a multimer via one or more kissing loops. In another aspect, the nucleic acid nanostructure further comprises one or more DNA or RNA staple strands. In another aspect, the nucleic acid nanostructure has a substantially cylindrical tube shape. In another aspect, the structuring sequence comprises a long hairpin along the inside edge of the cylindrical tube. In another aspect, the nucleic acid nanostructure has intramolecular folding capabilities without the need for helper nucleic acid strands. In another aspect, the structuring sequence is bound to the 5′-UTR or region of the 5′-UTR, wherein the 5′-UTR cannot bind to a ribosome or be degraded by a nuclease. In another aspect, the poly-A tail is located before the structuring sequence, after the structuring sequence, or at the 3′-end. In another aspect, one or more of the 5′-UTR and the 3′-UTR is derived from murine beta-globin. In another aspect, the 5′-UTR has a sequence that recruits RNA Helicase A (RHA). In another aspect, the sequence that recruits RNA Helicase A is from HIV 5′ leader sequence or human junD.

Another embodiment described herein is a method for selectively expressing a coding sequence in a target cell, the method comprising: delivering to a target cell a nanoparticle composition comprising a folded single-stranded nucleic acid nanostructure comprising: a 5′-untranslated region (UTR); a coding sequence; a structuring sequence that is complementary to the coding sequence and 5′-UTR or a region of the coding sequence and the 5′-UTR; a 3′-UTR; and a polyadenosine (poly-A) tail; wherein the structuring sequence is bound to the 5′-UTR and encapsulated in the nanostructure; wherein upon delivering the nanoparticle composition to the target cell, a target cell nucleic acid molecule binds the structuring sequence and displaces the 5′-UTR, thereby releasing the 5′-UTR for translation and expression of the coding sequence. In one aspect, the target cell nucleic acid molecule is associated with a virus or a disease. In another aspect, the nanoparticle composition comprises a plurality of therapeutic mRNAs.

Another embodiment described herein is a kit comprising: the composition of claim 1; a device for administering the composition of claim 1; a label or instructions for use; and packaging.

It will be apparent to one of ordinary skill in the relevant art that suitable modifications and adaptations to the compositions, formulations, methods, processes, kits, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

mRNA nanoparticles were generated using in vitro transcription off of a linearized plasmid molecule encoding a nanoparticle coding sequence and downstream structuring sequence (pOG-MCS, see below). Nanoparticles were purified of impurities and then thermally annealed in PBS or citrate buffer containing between 0 and 1 M NaCl by slowing cooling the solution from 65° C. to 15° C. at a rate of 1° C./15 minutes.

TABLE 1
Sequences (5′→3′)

| Name | Type | SEQ ID |
|---|---|---|
| HIV_RU5-6xHis-eGFP-OG-v6 | RNA | SEQ ID NO: 1 |

aggaauaaacuaguauucuucugguccccacagacucagagagaacccgccacccggggucgccguccuacacauu
guugugacgugcggcccagauucgaaucuguaauaaaaguuuucuuuucuucuauauccucagauuggcagugaga
ggagauuuuguucgugguguaggcuggccuacugggugggguugggauccggacugaauccguagaauuucuguac
aacauacCAUGGCACACCACCACCACCACCACCACauggugagcaagggcgaggagcuguucaccgggguggugcc
cauccuggucgagcuggacggcgacguaaacggccacaaguucagcguguccggcgagggcgagggcgaugccacc
uacggcaagcugacccugaaguucaucugcaccaccggcaagcugcccgugcccuggcccacccucgugaccaccc
ugaccuacggcgugcagugcuucagccgcuaccccgaccacaugaagcagcacgacuucuucaaguccgccaugcc
cgaaggcuacguccaggagcgcaccaucuucuucaaggacgacggcaacuacaagacccgcgccgaggugaaguuc
ggccccgugcugcugcccgacaaccacuaccugagcacccaguccgcccugagcaaagaccccaacgagaagcgcg
gagggcgacacccuggugaaccgcaucgagcugaagggcaucgacuucaaggaggacggcaacauccuggggcaca
agcuggaguacaacuacaacagccacaacgucuauaucauggccgacaagcagaagaacggcaucaaggugaacuu
caagauccgccacaacaucgaggacggcagcgugcagcucgccgaccacuaccagcagaacacccccaucggcgac
aucacaugguccugcuggaguucgugaccgccgccgggaucacucucggcauggacgagcuguacaaguaaugaua
auagaccgguaucguagucgcacgaccuggacacgccgaacuuccgcgggucggcugacgucgauggggugcgugc
aaaaaagaccuacgaagccagaguucguuccagugugaaagugcacaucacgaguugugccaaugcacguugcauc
gagggcugaagccgucuuaauauagacggcaccugaagagugauugauucgucuagaaauagacgaaucaugcuga
ucucaggugcucacuugauuaagacggcuguuuaucucgaugcucgcccucuuggcacaaucgaacuugugcacuu
cagcacgggaacgaacucuggcuucguaggucaaaaaagcacgcaagcauguaacgucagccuaacgcuugaaguu
cgcaggugugaggucgugcauguggucuaccgguucugcuugucuacuuguacagcucguccaugccagaucagca
cccggcgaaaaaaacgaaccgugagguagccauguagauacucugcucguugccuugccguaucagggcucaccag
gagcucagguagugguugucgggcagucacacugagccgucgauacaugcucuguucuaaaaaaaguggucgcacu
auagcacgcugucucguacaugguugucagaagucgcgaaguucaccuugaugccguucucuauuaucagggccau
gaucaagugacuguggcuuaccucacaguacucaaaaaagugccccaagauuugaccguccgaugaacuuacgaug
cccuucagcucgaugcgguggacuggggugucgccacucgugaugaccucguaagcguuacuguaguuuuauagug
ccuugaagaaaaaagugcgcucuugaucagagccuucgggcauggcggacuugggcggaucuggcugcuuggacua
cgacggguagcauaaacaggacugcacgagaguaucgaggguggcaaaucuugugggccaaaaaacgggcagcuug
ccgguggugcaaccuugaagaagggucagggucuuugggguggcgaacgugcaucccucgcacacaccugugaacu
uuuguacgagcacgucgcgugaucaagugaccaggaugggcccggguaauagaaaaaaaaCCCGGGugagcuggag
ccucgguggccuagcuucuugccccuugggccuccccccagccccuccuccccuuccugcacccguacccccgugg
ucuuugaauaaagucugagugggcggcaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaaaaaaaaaaaaaa
aaaaaaaaaaaaaaaa

| | | |
|---|---|---|
| junD_5UTR-6xHis-eGFP-OG-v6 | RNA | SEQ ID NO: 2 |

aggaauaaacuaguauucuucugguccccacagacucagagagaacccgccaccAGGAGCCGCCGCCAGUGGAGGG
CCGGGCGCUGCGGCCGCGGCCGGGGGGGGCGCAGGGCCGAGCGGACGGGGGGGCGCGGGCCCCCCGGGAGGCCGCG
GCCACUCCCCCCCGGGCCGGCGCGGCGGGGGAGGCGGAGGacCAUGGCACACCACCACCACCACCACCACauggug
agcaagggcgaggagcuguucaccgggguggugcccauccuggucgagcuggacggcgacguaaacggccacaagu
ucagcguguccggcgagggcgagggcgaugccaccuacggcaagcugacccugaaguucaucugcaccaccggcaa
gcugcccgugcccuggcccacccucgugaccacccugaccuacggcgugcagugcuucagccgcuaccccgaccac
augaagcagcacgacuucuucaaguccgccaugcccgaaggcuacguccaggagcgcaccaucuucuucaaggacg
acggcaacuacaagacccgcgccgaggugaaguucgagggcgacacccuggugaaccgcaucgagcugaagggcau
cgacuucaaggaggacggcaacauccuggggcacaagcuggaguacaacuacaacagccacaacgucuauaucaug
gccgacaagcagaagaacggcaucaaggugaacuucaagauccgccacaacaucgaggacggcagcgugcagcucg
ccgaccacuaccagcagaacacccccaucggcgacggccccgugcugcugcccgacaaccacuaccugagcaccca
guccgcccugagcaaagaccccaacgagaagcgcgaucacaugguccugcuggaguucgugaccgccgccgggauc
acucucggcauggacgagcuguacaaguaaugauaauagaccgguaucguagucgcacgaccuggacacgccgaac
uuccgcgggucggcugacgucgauggggugcgugcaaaaaagaccuacgaagccagaguucguuccagugugaaag
ugcacaucacgaguugugccaaugcacguugcaucgagggcugaagccgucuuaauauagacggcaccugaagagu
gauugauucgucuagaaauagacgaaucaugcugaucucaggugcucacuugauuaagacggcuguuuaucucgau
gcucgcccucuuggcacaaucgaacuugugcacuucagcacgggaacgaacucuggcuucguaggucaaaaaagca
cgcaagcauguaacgucagccuaacgcuugaaguucgcaggugugaggucgugcauguggucuaccgguucugcuu
gucuacuuguacagcucguccaugccagaucagcacccggcgaaaaaaacgaaccgugagguagccauguagauac
ucugcucguugccuugccguaucagggcucaccaggagcucagguagugguugucgggcagucacacugagccguc
gauacaugcucuguucuaaaaaaaguggucgcacuauagcacgcugucucguacaugguugucagaagucgcgaag
uucaccuugaugccguucucuauuaucagggccaugaucaagugacuguggcuuaccucacaguacucaaaaaagu
gccccaagauuugaccguccgaugaacuuacgaugcccuucagcucgaugcgguggacuggggugucgccacucgu
gaugaccucguaagcguuacuguaguuuuauagugccuugaagaaaaaagugcgcucuugaucagagccuucgggc
auggcggacuugggcggaucuggcugcuuggacuacgacggguagcauaaacaggacugcacgagaguaucgaggg
uggcaaaucuugugggccaaaaaacgggcagcuugccgguggugcaaccuugaagaagggucagggucuuuggggu
ggcgaacgugcaucccucgcacacaccugugaacuuuuguacgagcacgucgcgugaucaagugaccaggaugggc
ccggguaauagaaaaaaaaCCCGGGugagcuggagccucgguggccuagcuucuugccccuugggccuccccccag
ccccuccuccccuuccugcacccguacccccguggucuuugaauaaagucugagugggcggcaaaaaaaaaaaaaa
aaaaaaaaaaaaaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaa

| | | |
|---|---|---|
| eGFP-OG-v6 | RNA | SEQ ID NO: 3 |

aggaauaaacuaguauucuucugguccccacagacucagagagaacccgccaccauggugagcaagggcgaggagc
uguucaccgggguggugcccauccuggucgagcuggacggcgacguaaacggccacaaguucagcguguccggcga
gggcgagggcgaugccaccuacggcaagcugacccugaaguucaucugcaccaccggcaagcugcccgugcccugg
cccacccucgugaccacccugaccuacggcgugcagugcuucagccgcuaccccgaccacaugaagcagcacgacu
ucuucaaguccgccaugcccgaaggcuacguccaggagcgcaccaucuucuucaaggacgacggcaacuacaagac
ccgcgccgaggugaaguucgagggcgacacccuggugaaccgcaucgagcugaagggcaucgacuucaaggaggac
ggcaacauccuggggcacaagcuggaguacaacuacaacagccacaacgucuauaucauggccgacaagcagaaga
acggcaucaaggugaacuucaagauccgccacaacaucgaggacggcagcgugcagcucgccgaccacuaccagca
gaacacccccaucggcgacggccccgugcugcugcccgacaaccacuaccugagcacccaguccgcccugagcaaa
gaccccaacgagaagcgcgaucacaugguccugcuggaguucgugaccgccgccgggaucacucucggcauggacg
agcuguacaaguaaugauaauagaccgguaucguagucgcacgaccuggacacgccgaacuuccgcgggucggcug
acgucgauggggugcgugcaaaaaagaccuacgaagccagaguucguuccagugugaaagugcacaucacgaguug
ugccaaugcacguugcaucgagggcugaagccgucuuaauauagacggcaccugaagagugauugauucgucuaga
aauagacgaaucaugcugaucucaggugcucacuugauuaagacggcuguuuaucucgaugcucgcccucuuggca
caaucgaacuugugcacuucagcacgggaacgaacucuggcuucguaggucaaaaaagcacgcaagcauguaacgu
cagccuaacgcuugaaguucgcaggugugaggucgugcauguggucuaccgguucugcuugucuacuuguacagcu
cguccaugccagaucagcacccggcgaaaaaaacgaaccgugagguagccauguagauacucugcucguugccuug
ccguaucagggcucaccaggagcucagguagugguugucgggcagucacacugagccgucgauacaugcucuguuc
uaaaaaaaguggucgcacuauagcacgcugucucguacaugguugucagaagucgcgaaguucaccuugaugccgu
ucucuauuaucagggccaugaucaagugacuguggcuuaccucacaguacucaaaaaagugccccaagauuugacc
guccgaugaacuuacgaugcccuucagcucgaugcgguggacuggggugucgccacucgugaugaccucguaagcg
uuacuguaguuuuauagugccuugaagaaaaaagugcgcucuugaucagagccuucgggcauggcggacuugggcg
gaucuggcugcuuggacuacgacggguagcauaaacaggacugcacgagaguaucgaggguggcaaaucuuguggg
ccaaaaaacgggcagcuugccgguggugcaaccuugaagaagggucagggucuuugggguggcgaacgugcauccc
ucgcacacaccugugaacuuuuguacgagcacgucgcgugaucaagugaccaggaugggcccggguaauagaaaaa
aaaCCCGGGugagcuggagccucgguggccuagcuucuugccccuugggccuccccccagccccuccuccccuucc
ugcacccguacccccguggucuuugaauaaagucugagugggcggcaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaaaaaaaaaaaaaa
aaaaaaaaaaaaaaaaaaaaaaaaaaaaaaaaaaa

| | | |
|---|---|---|
| eGFP-OG-v7 | RNA | SEQ ID NO: 4 |

AGGAAAUAAGAGAGAAAAGAAGAGUAAGAAGAAAUAUAAGAGCCACCAUGGUGAGCAAGGGCGAGGAGCUGUUCAC
CGGGGUGGUGCCCAUCCUGGUCGAGCUGGACGGCGACGUAAACGGCCACAAGUUCAGCGUGUCCGGCGAGGGCGAG
GGCGAUGCCACCUACGGCAAGCUGACCCUGAAGUUCAUCUGCACCACCGGCAAGCUGCCCGUGCCCUGGCCCACCC
UCGUGACCACCCUGACCUACGGCGUGCAGUGCUUCAGCCGCUACCCCGACCACAUGAAGCAGCACGACUUCUUCAA
GUCCGCCAUGCCCGAAGGCUACGUCCAGGAGCGCACCAUCUUCUUCAAGGACGACGGCAACUACAAGACCCGCGCC
GAGGUGAAGUUCGAGGGCGACACCCUGGUGAACCGCAUCGAGCUGAAGGGCAUCGACUUCAAGGAGGACGGCAACA
UCCUGGGGCACAAGCUGGAGUACAACUACAACAGCCACAACGUCUAUAUCAUGGCCGACAAGCAGAAGAACGGCAU
CAAGGUGAACUUCAAGAUCCGCCACAACAUCGAGGACGGCAGCGUGCAGCUCGCCGACCACUACCAGCAGAACACC
CCCAUCGGCGACGGCCCCGUGCUGCUGCCCGACAACCACUACCUGAGCACCCAGUCCGCCCUGAGCAAAGACCCCA
ACGAGAAGCGCGAUCACAUGGUCCUGCUGGAGUUCGUGACCGCCGCCGGGAUCACUCUCGGCAUGGACGAGCUGUA
CAAGUAAUAGUGAGAAGUUGCCAUCGUAGUCGCACGACCUGGACACGCCGAACUUCCGCGGGUCGGCUGACGUCGA
UGGGGUGCGUGCAAAAAAGACCUACGAAGCCAGAGUUCGUUCCAGUGUGAAAGUGCACAUCACGAGUUGUGCCAAU
GCACGUUGCAUCGAGGGCUGAAGCCGUCUUAAUAUAGACGGCACCUGAAGAGUGAUUGAUUCGUCUAGAAAUAGAC
GAAUCAUGCUGAUCUCAGGUGCUCACUUGAUUAAGACGGCUGUUUAUCUCGAUGCUCGCCCUCUUGGCACAAUCGA
ACUUGUGCACUUCAGCACGGGAACGAACUCUGGCUUCGUAGGUCAAAAAAGCACGCAAGCAUGUAACGUCAGCCUA
ACGCUUGAAGUUCGCAGGUGUGAGGUCGUGCAUGUGGUCUGGCAACUCUGCUUGUUUACUUGUACAGCUCGUCCAU
GCCGGAUCAGCACCCGGCGAAAAAAACGAACUGUGAGGUAACCAUGUGGAUACUCUUCUCGUUGGCUUGCCGUCUC
AGGGCUCACCAGGUGCUCAGGUAGUGGUUGUCGGGCAGUCACACUGGGCCGUCGCUACAUGCUGUGUUCUAAAAAA
AGUGGUCGCACUAUAGCACGCUGCCUCGUACAAUGUUGUGAGAAGUCGUGAAGUUCACCUUGAUGCCGUUCUUUCU
CACUACGGCCAUGAUCAAGUGAUUGUGGCUUACCUCACUGUACUCAAAAAAGUGCCCCAAGAUUUGGCCGUCCUAU
GAACUUUCGAUGCCCUUCAGCUCGAUGCGGUGGACUGGGGUGUCGCCCCUCGUGAUCACCUCGGAAGCGUUAUUGU
AGUUGUAUAGUGCCUUGAAGAAAAAAGUGCGCUCUUGAUCAUAGCCUUCGGGCAUGGCGGACUUGAGCGGAUCUUG
CUGCUUCGACUACGAGGGGUAGCAUAAACAGCACUGCACGAGAGUAUCCAGGGUGGCAAAUCUUGUGGGCCAAAAA
ACGGGCAGCUUGCCGGUGGUGCAGCCUUGAAGCAGGGUCAGGGUCUUUGAGGUGGCAAACGUGCAGCCCUCGCCCA
CACCUGUGAACUUGUGUACGAGUACGUCGCCUGAUCAAGCGACCAGGAUGGGcccgggGCGGCCGCUUAAUUAAGC
UGCCUUCUGCGGGGCUUGCCUUCUGGCCAUGCCCUUCUUCUCUCCCUUGCACCUGUACCUCUUGGUCUUUGAAUAA
AGCCUGAGUAGGAAGAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAA

| | | |
|---|---|---|
| nanoLuciferase-OG v1 | RNA | SEQ ID NO: 5 |

aggAGGAAAUAAGAGAGAAAAGAAGAGUAAGAAGAAAUAUAAGAGCCACCGCCACCAUGGUCUUCACACUCGAAGA
UUUCGUUGGGGACUGGCGACAGACAGCCGGCUACAACCUGGACCAAGUCCUUGAACAGGGAGGUGUGUCCAGUUUG
UUUCAGAAUCUCGGGGUGUCCGUAACUCCGAUCCAAAGGAUUGUCCUGAGCGGUGAAAAUGGGCUGAAGAUCGACA
UCCAUGUCAUCAUCCCGUAUGAAGGUCUGAGCGGCGACCAAAUGGGCCAGAUCGAAAAAAUUUUUAAGGUGGUGUA
CCCUGUGGAUGAUCAUCACUUUAAGGUGAUCCUGCACUAUGGCACACUGGUAAUCGACGGGGUUACGCCGAACAUG
AUCGACUAUUUCGGACGGCCGUAUGAAGGCAUCGCCGUGUUCGACGGCAAAAAGAUCACUGUAACAGGGACCCUGU
GGAACGGCAACAAAAUUAUCGACGAGCGCCUGAUCAACCCCGACGGCUCCCUGCUGUUCCGAGUAACCAUCAACGG
AGUGACCGGCUGGCGGCUGUGCGAACGCAUUCUGGCGUAAUAGUGAUUAUAUCGGGCCCCGUCGAGGACACGAAAA
AAGAAGGUGCUGUUAGUUGGACAGGUACUAUCAUCUCAAGUCGAUAGUCCAAGUAGGUUUGAACCAUGCAUAGCUU
GUAUCAGGUCAUCGCCACCUUCAUUAGGUGUCUGAUCAGGGAAUCGCAAAAAACAUACCGACUUCCAUUAUGGGAC
ACGUCGCUUAUUCUUGGUAAGUAGAAGUUGCCAUCGUAGUCGCACGACCUACACACCUCGAACUUCAUCAUGUUGG
CUGACGUACUAACAGUGCGUGCAAAAAAGACCUACGAAGCCAGAGUUCGUUCCAGUGUGAAAGUGCACAUCACGAG
UUGUGCCAAUGCACGUUGCAUCGAGAUUUGGUCCCGUCUUAAAGGGAGGGCCACCUGAAUGGAAGUUGAUUCGUCU
AGAAAUAGACGAAUCAUGCUGAUCUCAGGUGCUCACUUGAUUAAGACGGCUGUUUAUCUCGAUGCAUUCUGAAUUG
GCACAACCGUCCGAGUGCACUUAUGAUAGUGAACGAACUCUGGCUUCGUAGGUCAAAAAAGCACGCAAGCAUGUAA
CGUCAGCCUAACGCUUGAAGUUCGCAGGUGUGAGGUCGUGCUUUUUCGAUGGCAACUUGGUUACUCCAAGAAUAAG
CGACGUGUCCCAUAGAUCAGCACGGUAUGAAAAAAGCGAUUCGUGAGGUAGACACCUAGAUACUCUGGCGAUGACU
UACGGACAGCUAUGCGCGAUGCCAACCUACUUGGACUAUCGACUUGAGUCACACUGACCUGUCCAUACAUGCUCAC
CUUCAAAAAACGUGUCCGCACUAUAGCCCGUCAUCUCGUACAAGGAAUGCUACACCACCGCCGCCAGCCGGUCACU
CCGUUGCUCUACUUAGGGAACAGAUCAAGUGACUCGGGGUUACCUCACAGCUCGUAAAAAAUUUUGUCAAGAUUUG
ACAGGGUGAAUCCUUUAGUGAUCUUUUUGCCGUCGAACACUAUGGUUCAGUCAUACGACUCGUGAUGAUAGUCUAA
GCGUUACGGCGUAAUUAUAGUGCCUACCAGAAAAAAAUAGUGUCUUGAUCAGUUAAAGUGAUGAUCAUCCACAGGG
GUUCGCACGUUAAAAAGGACUACGACCUGGCCCAUAAACAGGCCGCUCAGAGAGUAUCGCGGGAUGCAAAUCUUGA
UGUCGAAAAAAAGCCCAUUUUCACCGCUCAGGAACCUGUUACAGAUCGGAGCUGAUACAGCCCCGAGAACGUGCAU
CAAACUGACACACCUGUCCUGUUUUGUACGAGCGUCCAGGGUGAUCAAGUCUGUCUGUCGCCGCGGCCGCUUAAUU
AAGCUGCCUUCUGCGGGGCUUGCCUUCUGGCCAUGCCCUUCUUCUCUCCCUUGCACCUGUACCUCUUGGUCUUUGA
AUAAAGCCUGAGUAGGAAGAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAA

| | | |
|---|---|---|
| FluM1-HPV-OG v2 | RNA | SEQ ID NO: 6 |

AGGAAAUAAGAGAGAAAAGAAGAGUAAGAAGAAAUAUAAGAGCCACCAUGGCAGGAAAACCUAUUCCUAAUCCAUU
AUUAGGUUUAGAUUCUACAGAGGCCGCAGCAGCUAAGGGGAUCCUGGGCUUCGUGUUUACCCUCGCUGCCUACAAA
UCUGCUAUUGUGACAUUAACCUAUGCUGCCUACAACACAACUCCUAUUGUUCAUUUAAAGGCUGCCUACACACUUC
AAGAUGUUUCUUUAGAAGUAGCUGCCUACUAUGUGCAUGAAGGAAUUAGAACAUAUGCUGCCUACCAAGUUGAUUA
UUACGGCCUUUAUUAUGCUGCCUACAAUUUAGACACUGCUUCUACAACAUUAGCUGCCUACGUUCUUUUAUGUGUA
UGUCUGUUGAUUGCUGCCUACAAAUUGCCUCAACUUUGUACAGAAUUAGCUGCCUACACCAUACAUGAUAUUAUCC
UUGAAUGUGUUGCUGCCUACAUUAUACUGGAAUGCGUUUACUGUAAAGCUGCCUACUUUGCCUUUAGAGAUCUUUG
UAUUGUUGCUGCCUACACCACACUUGAGCAACAAUAUAAUAAAGCUGCCUACUAUAUGUUAGAUUUGCAACCUGAA
ACAACUGCUGCCUACAGAGCCCAUUAUAAUAUUGUGACAUUUGCUGCCUACCUUUUAAUGGGAACAUUAGGUAUUG
UUUGUCCCAUAUGUUCUCAAAAAUGAUAAGGAAUCGCAAAAAACAUACCGACUUCCAUUAUGGGACACGUCGCUUA
UUCUUGGUAAGUAGAAGUUGCCAUCGUAGUCGCACGACCUGAGGGUAACGAACUUCCACAUAAAGGCUGACGUCAG
UUGUUUGCGUGCAAAAAAGACCUACGAAGCCAGAGUUCGUUCCAGUGUGAAAGUGCACAUCACGAGUUGUGCCAAU
GCACGUUGCAUCGAGCAUGCACACCGUCUUAACUUUACAGGCACCUGAAUGGAAGUUGAUUCGUCUAGAAAUAGAC
GAAUCAUGCUGAUCUCAGGUGCUCACUUGAUUAAGACGGCUGUUUAUCUCGAUGCGCAGAUUUUUGGCACAAGCAA
UCAAGUGCACUUAUGGGCUCGAACGAACUCUGGCUUCGUAGGUCAAAAAAGCACGCAAGCAUGUAACGUCAGCCUA
ACGCUUGAAGUUCGCAGGUGUGAGGUCGUGCUAUGUUCUUGGCAACUAAAGGCAACCAAGAAUAAGCGACGUGUCC
CAUAGAUCAGCACGGUAUGAAAAAAGCGAUUCGUGAGGUAUUUUGAGAGAUACUCUGACAAACAAGGUUAAUGGUU
CCCAUGAGGCAAUAGGCAGCAAAUGUCACAAUAUUAUAUCACACUGUGUAGGCAGUACAUGCUUCAGGUUAAAAAA
CUAACAUGCACUAUACAGCUUUAUCUCGUACAUGCUCAAGCAACUUGGAGGCAGCAACAAUACAAAGAUCUCUUCU
ACUUAAGUAGGCAGUCAAGUGAUAAACGCAUACCUCACUAAUGUAAAAAAAAACACAUAAGAUUUGAAUAUCAUGU
GUUGUAUAGGCAGCUAAUUCUGUACAAAGUUUAAAAGGUUUGUAGGCACUCGUGAUCAGACAUAAAGCGUUAAGAA
CGUAGUAUAGUGCUGUUGUAAAAAAAGUGUCUACUUGAUCAGCAGCAUAAUAAAGGCCGUAAUAAUUGUGGUGUUA
GGCAGCAGACUACGAAAUUCCUUAUAAACAGUAGUAGGCAAGAGUAUCUAAAGAAACAAAUCUUAGUGUGUAAAAA
ACCUUUAAAUGAACAAUAGGAGUUGUAUGGUGGGCAGCAUAUACCUAAUUCACAAUAAACGUGCAGUAGGCAGCCA
CACCUGACACGAAGUGUACGAGCCCCUUAGCUGAUCAAGCCUCUGUAGAAUCcccgggGCGGCCGCUUAAUUAAGC
UGCCUUCUGCGGGGCUUGCCUUCUGGCCAUGCCCUUCUUCUCUCCCUUGCACCUGUACCUCUUGGUCUUUGAAUAA
AGCCUGAGUAGGAAGAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAA

| | | |
|---|---|---|
| pOG-MCS (Custom Cloning Vector) | DNA | SEQ ID NO: 7 |

atggcgcaggggatcaagctctgatcaagagacaggatgaggatcgtttcgcatgattgaacaagatggattgcac
gcaggttctccggccgcttgggtggagaggctattcggctatgactgggcacaacagacaatcggctgctctgatg
ccgccgtgttccggctgtcagcgcaggggcgcccggttctttttgtcaagaccgacctgtccggtgccctgaatga
actgcaagacgaggcagcgcggctatcgtggctggccacgacgggcgttccttgcgcagctgtgctcgacgttgtc
actgaagcgggaagggactggctgctattgggcgaagtgccggggcaggatctcctgtcatctcaccttgctcctg
ccgagaaagtatccatcatggctgatgcaatgcggcggctgcatacgcttgatccggctacctgcccattcgacca
ccaagcgaaacatcgcatcgagcgagcacgtactcggatggaagccggtcttgtcgatcaggatgatctggacgaa
gaacatcaggggctcgcgccagccgaactgttcgccaggctcaaggcgagcatgcccgacggcgaggatctcgtcg
tgacccatggcgatgcctgcttgccgaatatcatggtggaaaatggccgcttttctggattcatcgactgtggccg
gctgggtgtggcggaccgctatcaggacatagcgttggctacccgtgatattgctgaagaacttggcggcgaatgg
gctgaccgcttcctcgtgctttacggtatcgccgctcccgattcgcagcgcatcgccttctatcgccttcttgacg
agttcttctgaattattaacgcttacaatttcctgatgcggtattttctccttacgcatctgtgcggtatttcaca
ccgcatcaggtggcacttttcggggaaatgtgcgcggaacccctatttgtttatttttctaaatacattcaaatat
gtatccgctcatgagacaataaccctgataaatgcttcaataatagcacgtgctaaaacttcatttttaatttaaa
aggatctaggtgaagatcctttttgataatctcatgaccaaaatcccttaacgtgagttttcgttccactgagcgt
cagaccccgtagaaaagatcaaaggatcttcttgagatcctttttttctgcgcgtaatctgctgcttgcaaacaaa
aaaaccaccgctaccagcggtggtttgtttgccggatcaagagctaccaactctttttccgaaggtaactggcttc
agcagagcgcagataccaaatactgttcttctagtgtagccgtagttaggccaccacttcaagaactctgtagcac
cgcctacatacctcgctctgctaatcctgttaccagtggctgctgccagtggcgataagtcgtgtcttaccgggtt
ggactcaagacgatagttaccggataaggcgcagcggtcgggctgaacggggggttcgtgcacacagcccagcttg
gagcgaacgacctacaccgaactgagatacctacagcgtgagctatgagaaagcgccacgcttcccgaagggagaa
aggcggacaggtatccggtaagcggcagggtcggaacaggagagcgcacgagggagcttccagggggaaacgcctg
gtatctttatagtcctgtcgggtttcgccacctctgacttgagcgtcgatttttgtgatgctcgtcaggggggcgg
agcctatggaaaaacgccagcaacgcggcctttttacggttcctggccttttgctggccttttgctcacatgttct
tgctgcttcgcgatgtacgggccagatatacgcgttgacattgattattgactagttattaatagtaatcaattac
ggggtcattagttcatagcccatatatggagttccgcgttacataacttacggtaaatggcccgcctggctgaccg
cccaacgacccccgcccattgacgtcaataatgacgtatgttcccatagtaacgccaatagggactttccattgac
gtcaatgggtggagtatttacggtaaactgcccacttggcagtacatcaagtgtatcatatgccaagtacgccccc
tattgacgtcaatgacggtaaatggcccgcctggcattatgcccagtacatgaccttatgggactttcctacttgg
cagtacatctacgtattagtcatcgctattaccatggtgatgcggttttggcagtacatcaatgggcgtggatagc
ggtttgactcacggggatttccaagtctccaccccattgacgtcaatgggagtttgttttggcaccaaaatcaacg
ggactttccaaaatgtcgtaacaactccgccccattgacgcaaatgggcggtaggcgtgtacggtgggaggtctat
ataagcagagctctctggctaactagagaacccactgcttactggcttatcgaaattaatacgactcactataagg
AGGAAATAAGAGAGAAAAGAAGAGTAAGAAGAAATATAAGAGCCACCATGGaaaaaaaaaaCCCGGGGCGGCCGCT
TAATTAAGCTGCCTTCTGCGGGGCTTGCCTTCTGGCCATGCCCTTCTTCTCTCCCTTGCACCTGTACCTCTTGGTC
TTTGAATAAAGCCTGAGTAGGAAGaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaaaaaaaaaaaaaaaaaaa
aaaaaaaaaaaaaagaagagctctcgagtcgacctaggatcccttctactgggcggttttatggacagcaagcgaa
ccggaattgccagctggggcgccctctggtaaggttgggaagccctgcaaagtaaactggatggctttcttgccgc
caaggatctg

### Example 2

Production and Folding of eGFP-Encoding mRNA-OG Nanostructures

eGFP-encoding mRNA origami nanostructures (eGFP-OG) was created by cloning an eGFP coding sequence into the in vitro transcription vector pBspQI (Takara Biosciences), linearizing the resulting plasmid with BspQI, ethanol precipitating the resulting linear product, and performing in vitro transcription with cotranscriptional capping (CleanCap® AG, Trilink Biotechnology) as according to the NEB HiScribe T7 High Yield RNA Synthesis Kit (New England Biolabs) instructions for modified capped products. mRNA products were purified using the DNA Clean and Concentrator™-25 silica prep columns (Zymo Research) and resulting unfolded mRNA nanostructures were thermally annealed by diluting mRNA-OG to a concentration between 10-100 ng/μL in 1×PBS supplemented with 250 mM NaCl and adjusting their temperature from 65° C. to 15° C. at 1° C. per 15 minutes.

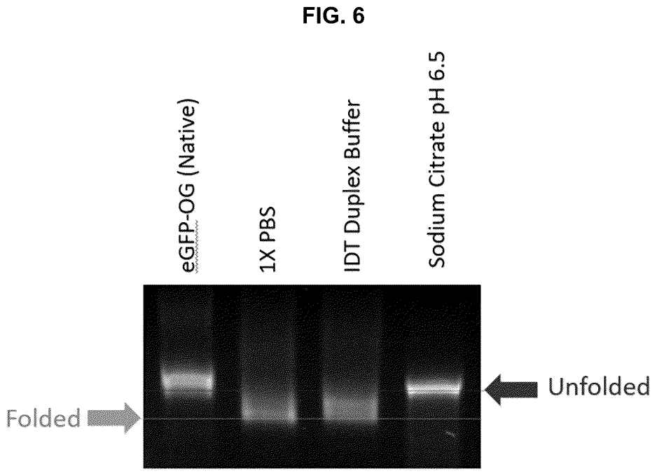

To assess the folding of the nanostructures, an agarose gel electrophoretic mobility shifty assay was performed (FIG. 6) and a relative increase in migration distance of thermally annealed nanostructure was observed relative to unfolded structure.

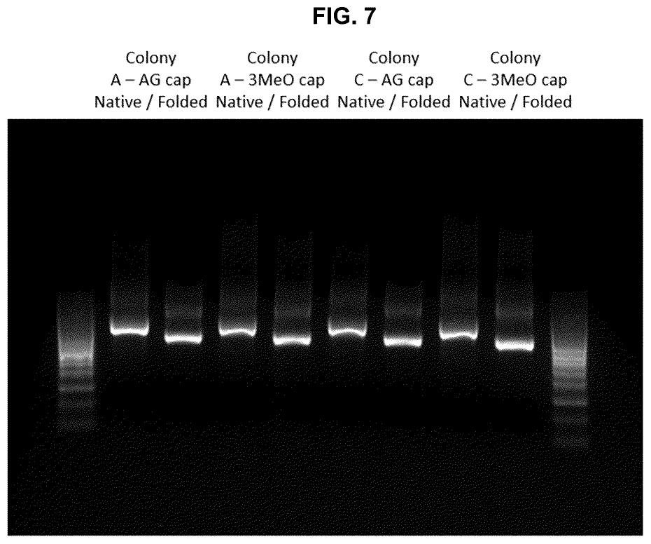

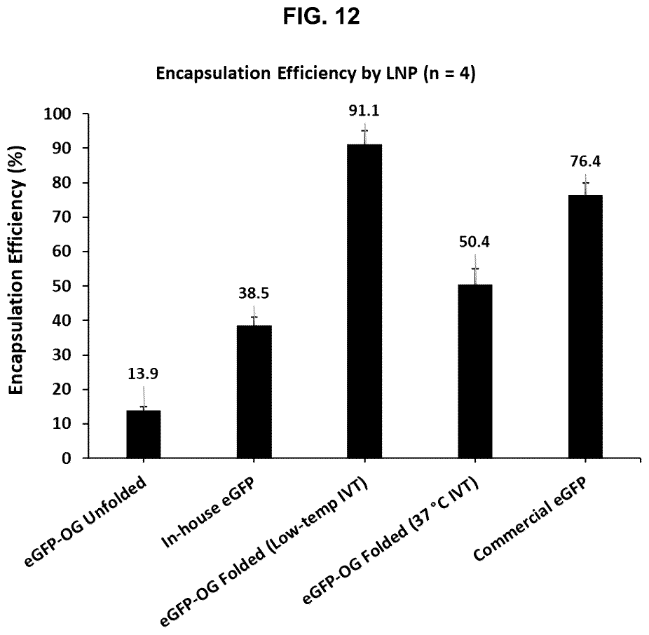

Agarose gel Electrophoretic Mobility Shift Assay (EMSA) was performed by casting a 1×TAE gel with no stain, loading 1 μg of sample per well, and running the gel at 90 V for 90 minutes at room temperature. Gels were post-stained using 3× GelRed in 1×TAE buffer for 30 minutes with gentle agitation before imaging on a GelDoc™ imaging platform (Biorad) (FIG. 7). To assess the encapsulation efficacy of the eGFP-OG, experiments using MessengerMax™ Lipofectamine™ transfection reagent (Thermo Fisher) were performed and the encapsulation efficiency was compared with unfolded eGFP-OG and unstructured mRNA coding for the same gene. eGFP-OG showed higher encapsulation efficiency (FIG. 12).

### Example 3

### Verifying Nanostructures Via Atomic Force Microscopy

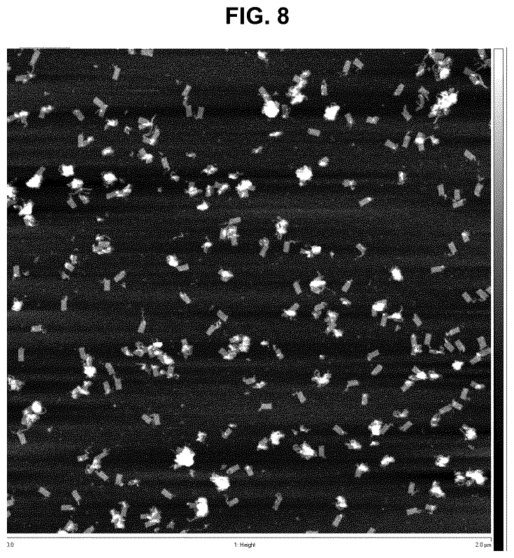

Thermally annealed and native mRNA origami was assessed correct structuring via atomic force microscopy (FIG. 8) as follows. First, 1 μg of unfolded eGFP-OG mRNA was either thermally annealed overnight as above or incubated in thermal annealing buffer at 4° C. overnight. Samples were diluted to ~20 nM in 1×TAE supplemented with MgCl₂ (4 mM) immediately prior to deposition of 5-15 μL onto a freshly cleaved mica surface. This volume was incubated for 5 minutes and then 35-45 μL of 1×TAE supplemented with 5 mM NiCl₂ was added on top. Sample was incubated for a further 5 minutes before being imaged on a Bruker MultiMode-8 AFM using ScanAsyst-Liquid+ tips.

### Example 4

Expression of eGFP-OG in HEK293T Cells

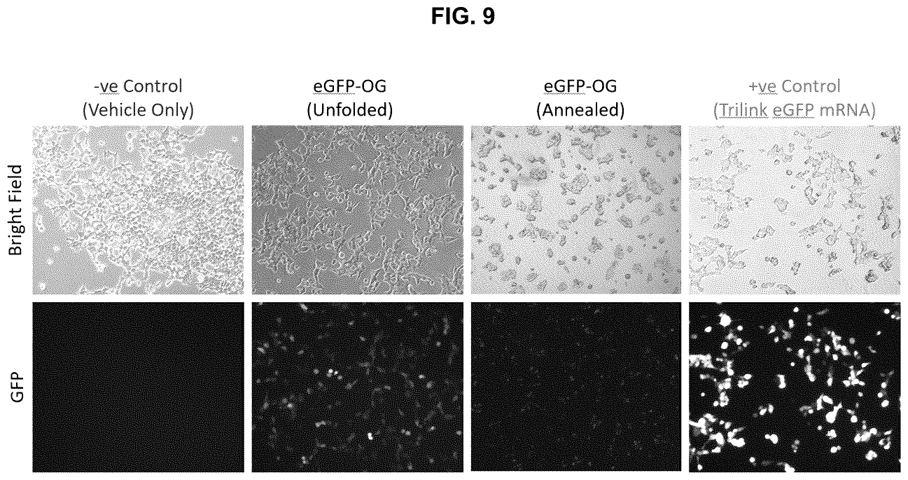

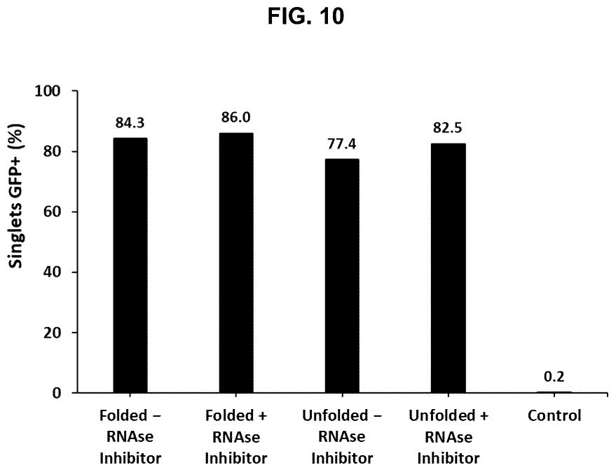

To test the expression of the nanoparticles, 2×10⁶ HEK293T cells were transfected with 0.75 μL MessengerMax™ Lipofectamine™ transfection reagent (Thermo Fisher) and 1 μg of either unfolded eGFP-OG, thermally annealed eGFP-OG, Trilink positive control eGFP mRNA, with or without the presence of RNAse Inhibitor (SUPERase OUT, ThermoFisher), or no RNA (vehicle control) according to manufacturer instructions. After 48 hours, cells were imaged (FIG. 9) and subjected to flow cytometry (FIG. 10) to assess eGFP expression.

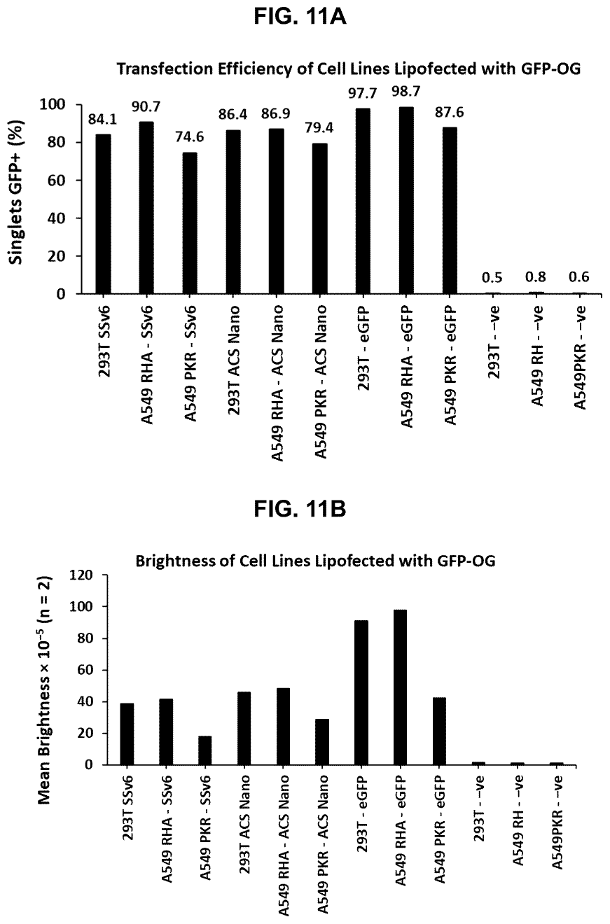

These data show expression of eGFP from both native structures and thermally annealed nanostructures (SSv6 in FIG. 11A) have similar levels of expression, indicating that the annealing process and annealed structure does not significantly inhibit cellular translation. Additionally, the nanostructures were tested in A549 cells with RNase H knock-out or PKR knock-down (FIG. 11B), which are both components of innate cellular dsRNA sensing and contribute to antiviral responses such as eIF4a phosphorylation and production of IFNγ. A significant difference was not observed in cells with innate immunity components missing, supporting the claim that folded mRNA-OG nanostructures are successfully translated.

What is claimed:

1. A nanoparticle composition comprising a folded single-stranded nucleic acid nanostructure comprising:
a 5′-untranslated region (5′-UTR);
a coding sequence;
a structuring sequence that is complementary to the coding sequence and the 5′-UTR or a region of the coding sequence and the 5′-UTR;
a 3′-untranslated region (3′-UTR); and
a polyadenosine (poly-A) tail;
wherein the structuring sequence is bound to the 5′-UTR and encapsulated in the nanostructure.

2. The composition of claim 1, wherein the coding sequence comprises a viral nucleic acid sequence, a bacterial nucleic acid sequence, or a parasitic nucleic acid sequence.

3. The composition of claim 2, wherein the viral nucleic acid sequence is derived from SARS CoV2, influenza virus, or HPV.

4. The composition of claim 1, wherein the coding sequence comprises a nucleic acid encoding one or more of CD40L, CD70, TLR4, and a neoantigen.

5. The composition of claim 1, wherein the coding sequence comprises a reporter gene selected from the group consisting of NanoLuc luciferase (Nluc), mCitrine, mCherry, mStrawberry, green fluorescent protein (GFP), enhanced green fluorescent protein (EGFP), yellow fluorescent protein (YFP), and red fluorescent protein (RFP).

6. The composition of claim 1, wherein the nucleic acid nanostructure comprises messenger RNA (mRNA), single-stranded RNA (ssRNA); single-stranded DNA (ssDNA), or a combination thereof.

7. The composition of claim 1, wherein the structuring sequence comprises one or more double crossover motifs, paranemic crossover motifs, kissing loops, or combinations thereof.

8. The composition of claim 1, wherein the composition comprises a plurality of folded nucleic acid nanostructures stacked in a multimer via one or more kissing loops.

9. The composition of claim 1, wherein the nucleic acid nanostructure further comprises one or more DNA or RNA staple strands.

10. The composition of claim 1, wherein the nucleic acid nanostructure has a substantially cylindrical tube shape.

11. The composition of claim 6, wherein the structuring sequence comprises a long hairpin along the inside edge of the cylindrical tube.

12. The composition of claim 1, wherein the poly-A tail is located before the structuring sequence, after the structuring sequence, or at the 3′-end.

13. The composition of claim 1, wherein one or more of the 5′-UTR and the 3′-UTR is derived from murine beta-globin.

14. The composition of claim 1, wherein the 5′-UTR has a sequence that recruits RNA Helicase A (RHA).

15. The composition of claim 12, wherein the sequence that recruits RNA Helicase A is from HIV 5′ leader sequence or human junD.

16. A method for selectively expressing a coding sequence in a target cell, the method comprising:
delivering to a target cell a nanoparticle composition comprising a folded single-stranded nucleic acid nanostructure comprising:
a 5′-untranslated region (UTR);
a coding sequence;
a structuring sequence that is complementary to the coding sequence and 5′-UTR or a region of the coding sequence and the 5′-UTR;
a 3′-UTR; and
a polyadenosine (poly-A) tail;
wherein the structuring sequence is bound to the 5′-UTR and encapsulated in the nanostructure;
wherein upon delivering the nanoparticle composition to the target cell, a target cell nucleic acid molecule binds the structuring sequence and displaces the 5′-UTR, thereby releasing the 5′-UTR for translation and expression of the coding sequence.

17. The method of claim 16, wherein the target cell nucleic acid molecule is associated with a cancer, a viral infection, a bacterial infection, or a parasitic infection.

18. The method of claim 16, wherein the nanoparticle composition comprises a plurality of therapeutic mRNAs stacked in a multimer via one or more kissing loops for stoichiometric co-delivery.

19. A kit comprising:
the composition of claim 1;
a device for administering the composition of claim 1;
a label or instructions for use; and
packaging.

20. The kit of claim 19, wherein the device for administering comprises a syringe, nebulizer, or lipid nanoparticle delivery system.
